(19) United States
(12) Patent Application Publication
(10) Pub. No.: US 2023/0042236 A1
(43) Pub. Date: Feb. 9, 2023

(54) LOCKOUT MECHANISM FOR SINGLE USE DEVICE

(71) Applicant: BOSTON SCIENTIFIC SCIMED, INC., Maple Grove, MN (US)

(72) Inventor: Michael S. H. Chu, Brookline, MA (US)

(73) Assignee: BOSTON SCIENTIFIC SCIMED, INC., Maple Grove, MN (US)

(21) Appl. No.: 17/879,467

(22) Filed: Aug. 2, 2022

Related U.S. Application Data
(60) Provisional application No. 63/228,807, filed on Aug. 3, 2021.

(51) Int. Cl. A61B 90/00

(52) U.S. Cl. CPC A61B 90/03; A61B 2090/0814; A61B 2090/037

(57) ABSTRACT

Single use medical devices and mechanisms for preventing the re-use thereof. A single use medical device may include a housing defining an insertion slot and a lockout mechanism. The lockout mechanism may comprise a biasing mechanism and a movable slide element positioned adjacent to the insertion slot and the biasing mechanism, the slide element movable between a pre-use configuration, an in-use configuration, and a post-use configuration. The slide element may be actuated via an insertion member removably inserted into the insertion slot.

### CROSS-REFERENCE TO RELATED APPLICATIONS

This application claims the benefit of U.S. Provisional Patent Application Ser. No. 63/228,807, filed Aug. 3, 2021, which is incorporated herein by reference.

### TECHNICAL FIELD

The disclosure is directed to single use medical devices. More particularly, the disclosure is directed systems and methods for preventing reuse of a single use medical device.

### BACKGROUND

Some medical devices are intended to be single use devices (SUD). Even though SUDS are intended to be used on a single patient, they may be made durable which may allow the SUD to be capable of use in more than one patient. Of the known medical devices, systems, and methods, each has certain advantages and disadvantages. There is an ongoing need to provide alternative medical devices. For example, what may be desirable are systems and methods which prevent a SUD from being used more than one time.

### SUMMARY

The disclosure is directed to systems and methods for preventing reuse of a single use device.

A first example is a single use medical device including a housing defining an insertion slot and a lockout mechanism. The lockout mechanism includes a biasing mechanism and a movable slide element positioned adjacent to the insertion slot and the biasing mechanism. The slide element is movable between a pre-use configuration, an in-use configuration, and a post-use configuration. The slide element is actuated via an insertion member removably inserted into the insertion slot.

Alternatively or additionally to any of the examples above, in another example, when the slide element is in the post-use configuration, the slide element is positioned at least partially within the insertion slot.

Alternatively or additionally to any of the examples above, in another example, when the slide element is in the pre-use configuration, the slide element mechanically engages a portion of the housing.

Alternatively or additionally to any of the examples above, in another example, when the slide element is in the in-use configuration, the slide element mechanically engages the insertion member

Alternatively or additionally to any of the examples above, in another example, the biasing mechanism is configured to exert a force on the slide element.

Alternatively or additionally to any of the examples above, in another example, in the absence of a stop mechanism, the biasing mechanism is configured to bias the slide element to the post-use configuration.

Alternatively or additionally to any of the examples above, in another example, when the slide element is in the post-use configuration, the slide element is visible through the insertion slot.

Alternatively or additionally to any of the examples above, in another example, the single use device includes a start post configured to engage a start button on a multi-use device.

Alternatively or additionally to any of the examples above, in another example, the single use device includes an elongated tip extending from an end of the slide element.

Alternatively or additionally to any of the examples above, in another example, the elongated tip is configured to disable a component of the single use device when the slide element is in the post-use configuration.

Alternatively or additionally to any of the examples above, in another example, insertion of the insertion member is configured to move the slide element from the pre-use configuration to the in-use configuration.

Alternatively or additionally to any of the examples above, in another example, the biasing mechanism comprises a compression spring.

Alternatively or additionally to any of the examples above, in another example, the biasing mechanism comprises a torsion spring.

Alternatively or additionally to any of the examples above, in another example, the single use device comprises a cassette for use in a fluid management system.

Alternatively or additionally to any of the examples above, in another example, the single use device comprises a laser fiber, an electrocautery plug, an electric plug, an optical plug, a fluid plug, or a single use scope shaft.

Another example is a medical device system including a multi-use device and a single use device. The multi-use device includes a housing defining a cavity and an insertion member. The single use device is configured to be removably coupled with the multi-use device. The single use device includes a housing defining an insertion slot and a lockout mechanism. The lockout mechanism includes a biasing mechanism and a movable slide element positioned adjacent to the insertion slot and the biasing mechanism. The slide element is movable between a pre-use configuration, an in-use configuration, and a post-use configuration. When the slide element is in the post-use configuration, the single use device is prevented from being functionally assembled with the multi-use device.

Alternatively or additionally to any of the examples above, in another example, insertion of the insertion member into the insertion slot is configured to move the slide element from the pre-use configuration to the in-use configuration and subsequent removal of the insertion member from the insertion slot is configured to move the slide element form the in-use configuration to the post-use configuration.

Another example is a medical device system including a multi-use device and a single use device configured to be removably coupled with the multi-use device. The multi-use device includes a housing defining a cavity, an insertion member, and a start button. The single use device includes a housing defining an insertion slot, a start post, and a lockout mechanism. The lockout mechanism includes a biasing mechanism and a movable slide element positioned adjacent to the insertion slot and the biasing mechanism. The slide element is movable between a pre-use configuration, an in-use configuration, and a post-use configuration. The slide element is moved from the pre-use configuration to the in-use configuration upon insertion of the insertion member into the insertion slot. When the slide element is in the post-use configuration, the single use device is prevented from being functionally assembled with the multi-use device.

Alternatively or additionally to any of the examples above, in another example, when the slide element is in the in-use configuration, the start post of the single use device engages the start button of the multi-use device.

Alternatively or additionally to any of the examples above, in another example, when the start post engages the start button, a timer is activated.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view of an illustrative fluid management system;

FIG. 2 is a schematic view of a portion of an illustrative peristaltic pump;

FIG. 3 is a perspective view of an illustrative pump console;

FIG. 4 is a side view of the illustrative pump console of FIG. 3;

FIG. 5 is a partially exploded perspective view of an illustrative single use cassette;

FIG. 6 is a front view of a base portion of the illustrative cassette of FIG. 5 with some components removed;

FIG. 7A is a top view of an illustrative slide element;

FIG. 7B is a side view of the illustrative slide element of FIG. 7A;

FIG. 8 is a front view of the base portion of the illustrative cassette of FIG. 5 in a pre-use configuration;

FIG. 9 is a front view of the illustrative single use device or cassette of FIG. 5 as it is being inserted into a pump console;

FIG. 10 is a front view of the illustrative single use device or cassette of FIG. 5 fully inserted into a pump console;

FIG. 11 is a front view of the illustrative single use device or cassette of FIG. 5 as it is being removed from a pump console;

FIG. 12 is a front view of the base portion of the illustrative cassette of FIG. 5 in a post-use configuration;

FIG. 13A is a top view of another illustrative slide element;

FIG. 13B is a top view of the illustrative slide element of FIG. 13A in a post-use configuration;

FIG. 14 is a schematic side view of another illustrative lockout mechanism;

FIG. 15 is a schematic top view of the illustrative lockout mechanism of FIG. 14;

FIG. 16 is an exploded perspective view of the illustrative lockout mechanism of FIG. 14;

FIG. 17 is a cross-sectional view of the illustrative lockout mechanism of FIG. 14 assembled with a pump console;

FIG. 18 is a cross-sectional view of the illustrative lockout mechanism of FIG. 14 in a post-use configuration with a pump console;

FIG. 19 is a schematic side view of another illustrative lockout mechanism in a pre-use configuration;

FIG. 20 is a schematic side view of the illustrative lockout mechanism of FIG. 19 in a post-use or disabled configuration;

FIG. 21 is a perspective view of another illustrative lockout mechanism in a pre-use configuration;

FIG. 22 is a perspective view of the illustrative lockout mechanism of FIG. 21 in a post-use or disabled configuration;

FIG. 23 is a side view of an illustrative single use laser fiber in a pre-use configuration;

FIG. 24 is a side view of the illustrative single use laser fiber of FIG. 23 in a use configuration; and

FIG. 25 is a side view of the illustrative single use laser fiber of FIG. 23 in a post-use configuration.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

The terms “proximal” and “distal” as used herein are intended to refer to a direction toward (proximal) and away from (distal) a user of the device. The terms top, bottom, back, front, first, and second are relative terms used merely to aid in discussing the drawings, and are not meant to be limiting in any manner.

Some medical devices are intended to be single use devices (SUD). Even though SUDs are intended to be used on a single patient, they may be made durable which may allow the SUD to be capable of use in more than one patient. However, the use of a device which has been designated as a single use device more than one time may risk patient to patient contamination and failures due to wear and tear conditions. The systems and methods described herein may create a mechanical lockout such that a SUD will no longer mate with a receiving device. For example, a single use cassette for use with a peristaltic fluid management system (FMS) may include a mechanical lockout that prevents a used cassette from being fully inserted into an insertion slot. In another example, a single use laser fiber may include a lockout mechanism that prevents a used laser fiber from being connected with another part of a device, such as, but not limited to a modular scope. However, other single use devices, such as, but not limited to, electrocautery plugs, electric plugs, optical plugs, fluid plugs, or other plugs for connecting one modular part to another part of a device may include lockout mechanisms. Generally, the lockout mechanism may prevent an insertion member of the reusable device from being fully inserted into an insertion slot of the SUD such that the SUD no longer properly mates with the reusable component after a single use. In some cases, the lockout mechanism may prevent a start button from being engaged and/or activated. Alternatively, or additionally, the lockout mechanism may be used to physically damage or disable the SUD to prevent use.

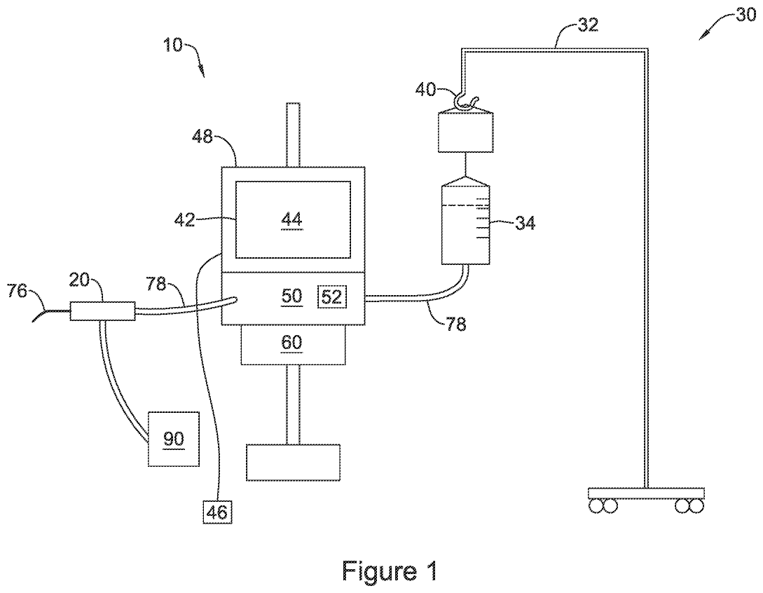

FIG. 1 is a schematic view of an illustrative fluid management system 10 that may be used in an endoscopic procedure. The fluid management system 10 may be coupled to a surgical device 20 that allows flow of fluid therethrough and includes a pressure sensor. An illustrative surgical device may be a LithoVue™ scope device, or other endoscope. In an illustrative embodiment, the device 20 may include a temperature sensor to provide intracavity temperature feedback to the fluid management system 10, a pressure sensor to provide intracavity pressure feedback to the fluid management system 10, and/or a camera to provide visual feedback to the fluid management system 10. As will be described in more detail herein, a single use cassette may be used with the fluid management system 10. Some illustrative fluid management systems 10 are described in described in commonly assigned U.S. Patent Publication Number 2020/0405955, titled DETECTION OF AN ENDOSCOPE TO A FLUID MANAGEMENT SYSTEM, and in commonly assigned U.S. patent application Ser. No. 17/186,989, titled ADAPTIVE PRESSURE CONTROL FILTER FOR A FLUID MANAGEMENT SYSTEM and filed on Feb. 26, 2021, the disclosures of which are hereby incorporated by reference

Briefly, the fluid management system 10 may include a pump system 50 configured to transfer fluid from a fluid bag 34 to the medical device 20. In some cases, the fluid may pass through a heating system 60 prior to entering the medical device 20. The flow of fluid, pressure of the fluid, temperature of the fluid, and other operational parameters may be controlled by or at least partially controlled by a main processing device 48 including a display screen 44. The main processing device 48 may be in electronic communication (e.g., wired or wireless) with the medical device 20, the pump system 50, and/or the heating system 60 to provide control commands and/or to transfer or receive data therebetween. The main processing system 48 may then use the data received from the medical device 20 to control operational parameters of the pump system 50 and/or the heating system 60.

The fluid management system 10 also includes a fluid management unit 30. An illustrative fluid management unit 30 may include one or more fluid container supports, such as fluid bag hangers 32, each of which supports one or more fluid bags 34. The fluid bag hanger 32 extends from the pole 36 and may include one or more hooks 40 from which one or more fluid bags 34 may be suspended.

The fluid management system 10 may also include one or more user interface components such as a touch screen interface 42. The touch screen interface 42 includes a display screen 44 and may include switches or knobs in addition to touch capabilities. The touch screen interface 42 allows the user to input/adjust various functions of the system 10 such as, for example flow rate, pressure or temperature. It is contemplated that other systems configured to receive user input may be used in place of or in addition to the touch screen interface 42. Some illustrative display screens 44 and other user interface components are described in described in commonly assigned U.S. Patent Publication Number 2018/0361055, titled AUTOMATED FLUID MANAGEMENT SYSTEM, the disclosure of which is hereby incorporated by reference.

The touch screen interface 42 may be operatively connected to or a part of the main processing device 48. The main processing device 48 may be a computer, tablet computer, or other processing device. The main processing device 48 may be operatively connected to one or more system components such as, for example, the pump system 50, the heating system 60 and a fluid deficit management system. The main processing device 48 is capable of and configured to perform various functions such as calculation, control, computation, display, etc. The main processing device 48 is also capable of tracking and storing data pertaining to the operations of the management system 10 and each component thereof.

In order to adjust the rate of flow of fluid through the system 10, the fluid management unit 30 may include one or more pressurization devices such as a pump 52. An illustrative pump 52 may be a peristaltic pump, or roller pump. As will be described in more detail herein, the pump 52 may include at least a pump console, which contains a motor and a motor shaft, and a single use cassette, which contains a flexible tube and a rotor. The pump 52 may be electrically driven and may receive power from a line source such as a wall outlet or an external or internal electrical storage device such as a disposable or rechargeable battery. The peristaltic pump 52 may operate at any desired speed sufficient to deliver fluid at a target pressure such as, for example, 5 mmHg to 50 mmHg. As noted previously, the pump 52 may be automatically adjusted based on, for example, pressure and temperature readings within the patient and/or visual feedback from the medical device 20. The pump 52 may also be manually adjusted via, for example, foot pedal 46, the touch screen interface 42, or a separate fluid controller.

In some embodiments, the medical device 20 may be a ureteroscope such as a LithoVue™ scope. However, other medical devices, such as another endoscope, may be used in addition to or in place of a ureteroscope. The medical device 20 delivers fluid from the fluid management system 10 to the target tissue via an elongate or scope shaft 76. The elongate shaft 76 may include one or more working lumens for receiving a flow of fluid or other medical devices therethrough. The medical device 20 is connected to the fluid management system 10 via one or more supply line(s) 78 (e.g., a tube). The medical device 20 may also include a drainage port which may be connected to a drainage system 90. Some illustrative drainage systems 90 are described in described in commonly assigned U.S. Patent Publication Number 2018/0361055, titled AUTOMATED FLUID MANAGEMENT SYSTEM, the disclosure of which is hereby incorporated by reference.

The fluid management system 10 may utilize small diameter pump tubing 78 to connect various components. Illustrative tubing 78 for irrigation procedures may be less than or equal to 1/16 inches in diameter. However, it will be understood that tubing size may vary based on the application. Tubing may be disposable and provided sterile and ready to use. Different types of tubing may be used for various functions within the fluid management system 10. For example, one type of tubing may be used for fluid heating and fluid flow control to the device 20 while another type of tubing may be used for irrigation within the body.

In an illustrative embodiment, the fluid management system 10 may optionally include a fluid warming system 60 for heating fluid to be delivered to the patient. Some illustrative heater systems 60 are described in described in commonly assigned U.S. Patent Publication Number 2018/0361055, titled AUTOMATED FLUID MANAGEMENT SYSTEM, the disclosure of which is hereby incorporated by reference.

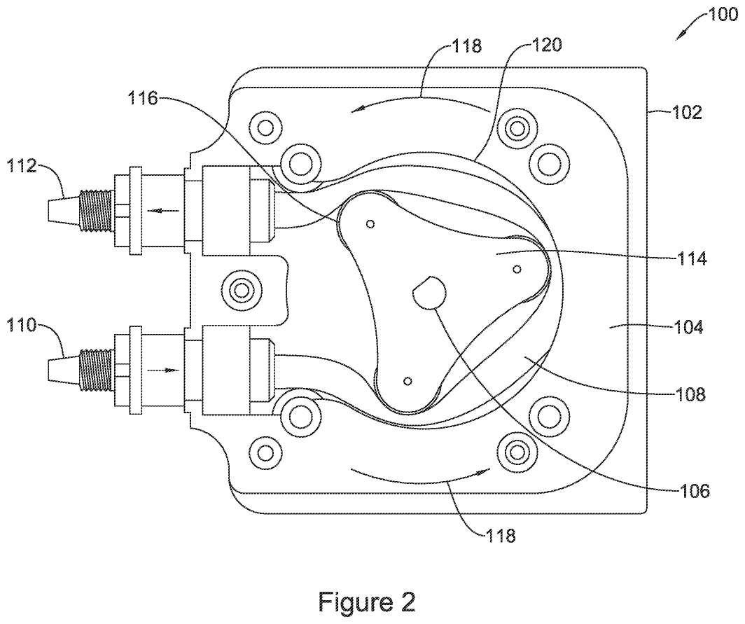

FIG. 2 is a schematic view of a portion of an illustrative peristaltic pump 100, or roller pump, that may be used with a fluid management system 10. While the pump 100 is described as a roller pump, the pump 100 may take other forms, as desired. The pump 100 may be a positive displacement pump used for pumping a variety of fluids including, but not limited to, saline. The pump 100 may include a pump console 102 and a cassette 104. The cassette 104 may be a filter cassette, a heater cassette, a chiller cassette, an electronic cassette, or the like. The cassette 104 may be a single use device (SUD) cassette 104 while the pump console 102 may be reused with different cassettes 104 for multiple procedures. The cassette 104 may be configured to be removably secured within the pump console 102 such that multiple different cassettes 104 may be used with a single pump console 102. Among other features, the pump console 102 may include a motor (not explicitly shown) and a motor shaft 106.

The cassette 104 may include a flexible tube 108 for conveying fluid from an inlet 110 to an outlet 112. The inlet 110 may be fluidly coupled to a fluid source, such as, but not limited to, a fluid bag 34, while the outlet 112 may be fluidly coupled to a medical device 20. An intermediate portion of the flexible tube 108 may be fitted inside a circular portion 120 of the cassette 104. The cassette 104 may further include a rotor 114 having a plurality of wipers or rollers 116 attached to the external circumference of the rotor 114. The cassette 104 may be configured to fit or nest into the pump console 102 such that the rotor 114 will align and mate with the motor shaft 106 in order to transfer the torque from the motor to the rotor 114. The fluid to be pumped is contained in the tube 108. As the rotor 114 rotates, the rollers 116 compress the tube 108 as they rotate by. The part of the tube 108 under compression is closed or reduced, forcing the fluid to move through the tube 108. Additionally, as the tube 108 opens to its natural state after the rollers 116 pass, more fluid is drawn into the tube 108, In some examples, there may be two or more rollers 116 compressing the tube 108, trapping a body of fluid between them. The body of fluid is transported through the tube 108 and toward the pump outlet 112, as shown at arrows 118. The pump 100 may run continuously, or it may be indexed through partial revolutions to deliver smaller amounts of fluid. In some cases, the fluid that is pumped through the cassette 104 is heated within the cassette 104 via radiofrequency heated metal plates or other heating mechanisms.

While the SUD cassette 104 may be made durable enough for more than one use, the cassette 104 may be designed and intended for use in a single patient and/or a predetermined length of time. Reuse of single use devices may risk patient to patient contamination and/or failures due to the wear and tear. Thus, it may be desirable to provide the cassette 104 and/or pump console 102 with a lockout mechanism that prevents full insertion of a previously used cassette 104 into the pump console 102. While the lockout mechanism is described with respect to a cassette 104, the lockout mechanism is not so limited. The lockout mechanism may be used in other single use devices.

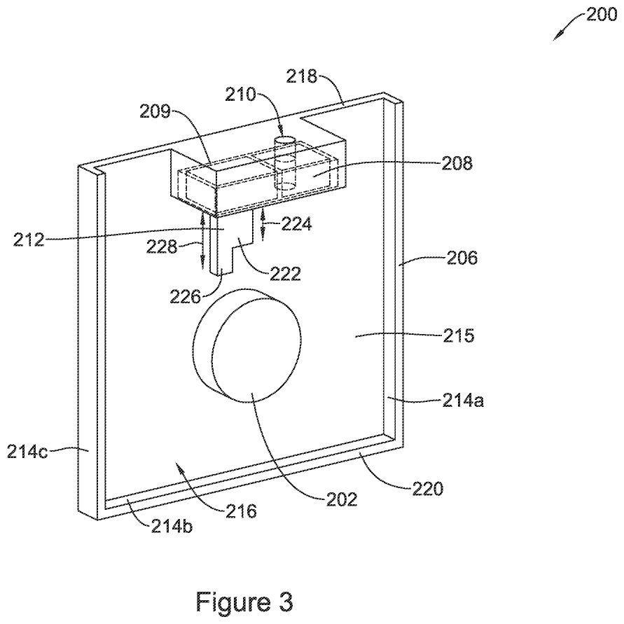

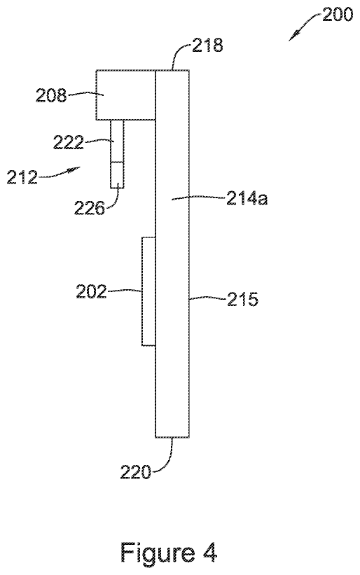

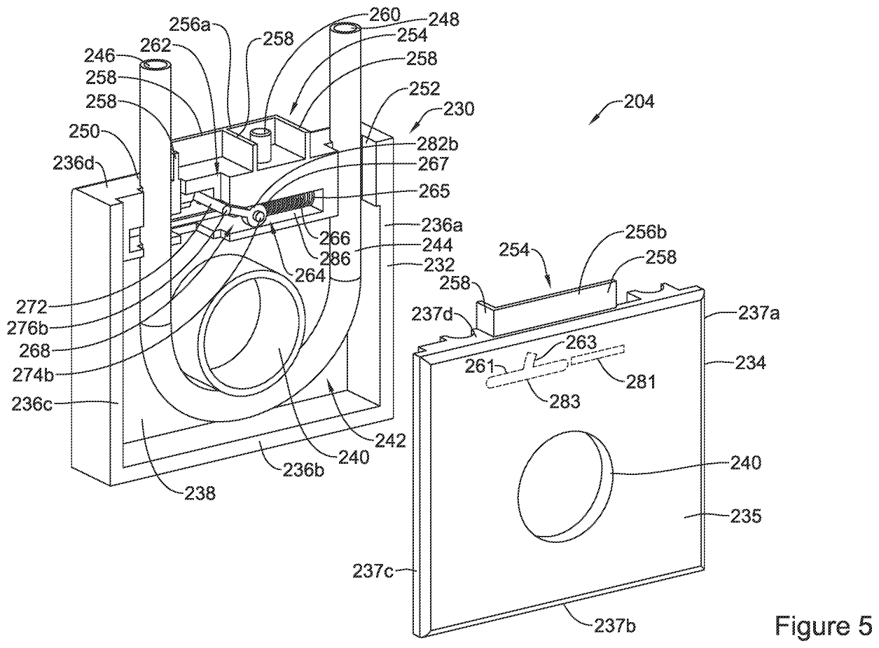

FIG. 3 is a perspective view of an illustrative pump console 200 for use with a single use cassette 204 (see, for example, FIG. 5) which may be similar in form and function to cassette 104. FIG. 4 is a side view of the illustrative pump console 200. The pump console 200 may include a motor shaft 202 extending from the housing 206 thereof. While not explicitly shown, the motor shaft 202 is operably coupled to a motor disposed in or adjacent to the housing 206. The housing 206 may include a first lateral sidewall 214a, a bottom sidewall 214b, and a second lateral sidewall 214c (collectively, 214). The sidewalls 214 together with a back plate 215 are configured to form a cavity or nest 216 for receiving a cassette 204 therein. In some cases, the sidewalls 214 may extend about three lateral edges of the housing 206. However, the sidewalls 214 may extend about fewer than three or more than three edges of the housing 206, as desired.

The housing 206 may further include a protruding engagement member 208 extending therefrom. The engagement member 208 may be configured to receive a portion of the cassette 204 therein, as will be described in more detail herein. For example, the engagement member 208 may include a plurality of recesses 209 that are configured to receive mating sidewalls of a cage of the cassette 204. The engagement member 208 may be positioned adjacent to a first or top edge 218 of the housing 206, although this is not required. In some cases, the engagement member 208 may house additional components for the pump. For example, the engagement member 208 may include a start button 210. The start button 210 may be configured to electrically and/or mechanically engage a start post in the cassette 204. It is contemplated that the pump will not function if the start button 210 is not engaged with a mating start post.

An insertion member or insertion post 212 may extend from a bottom edge of the engagement member 208 and towards a bottom edge 220 of the housing 206. A bottom, or second end of the insertion member 212 may be generally “L” shaped. The insertion member 212 may have a first portion 222 having a first length 224 and a second portion 226 having a second length 228. The first length 224 may be less than the second length 228. As will be described in more detail herein, the insertion member 212 may be configured to engage and activate a lockout mechanism on the cassette 204. Activation of the lockout mechanism may prevent reuse of the cassette 204.

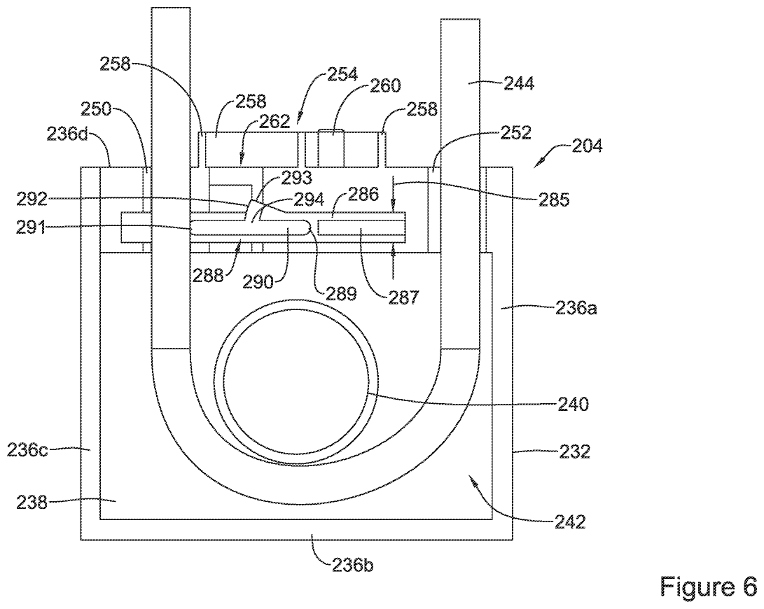

FIG. 5 is a partially exploded perspective view of an illustrative single use cassette 204. FIG. 6 is a front view of a base 232 portion of the illustrative cassette 204 of FIG. 5 with some components removed. In FIGS. 5 and 6, a rotor is not illustrated but rather is represented by a rotor hole 240 which receives the motor shaft 202 of the pump console 200. However, it should be understood that the cassette 204 may include a rotor similar in form and function to the rotor 114 described above. The cassette 204 may include a housing 230 having a base 232 and a covering 234. The base 232 may be a base configured to house the components of the cassette 204 while the covering 234 may be a lid or covering configured to secure the components within the base 232. In some cases, the covering 234 may be removably secured to the base 232. In other cases, the covering 234 may be fixedly secured to the base 232. In yet other examples, the first and second portions 232, 234 may be formed as a single monolithic structure.

The covering 234 may include a front plate 235 and a plurality of sidewalls 237a, 237b, 237c, 237d (collectively, 237). While not explicitly shown, the covering 234 may include features configured to secure the covering 234 to the base 232. The base 232 may include a plurality of sidewalls 236a, 236b, 236c, 236d (collectively, 236), which together with a back plate 238 form a cavity or recess 242 for receiving or holding additional components of the cassette 204. The cassette 204 may include a flexible tube 244 for conveying fluid from an inlet 246 to an outlet 248. The flexible tube 244 may enter the cavity 242 of the housing 230 via a first opening 250 in a top sidewall 236d and exit the cavity 242 via a second opening 252 in the top sidewall 236d. The tubing 244 may extend along an outer perimeter of the rotor hole 240 such that rotation of the rotor (not explicitly shown) moves fluid through the tube 244.

A cage 254 may extend away from a top edge of the housing 230. In some cases, a portion 256a of the cage 254 may extend from the top sidewall 236d of the base 232 of the housing 230 and a second portion 256b of the cage 254 may extend from the top sidewall 237d of the covering 234 of the housing 230. This is just one example. In some cases, the cage 254 may extend solely from either of the first or second portions 232, 234 of the housing 230. The cage 254 may include sidewalls 258 forming two generally rectangular boxes. The walls 258 of the cage 254 may be sized and shaped to mate with corresponding recesses 209 in the engagement member 208 of the pump console 200. Further, the walls 258 of the cage 254 may be configured such that assembly of the cassette 204 with the pump console 200 is limited to a predetermined orientation.

The cage 254 may be further configured to surround and/or protect a start post 260 and an insertion slot 262. As will be described in more detail herein, the insertion slot 262 is configured to receive the insertion member 212 of the pump console 200. When the cassette 204 is fully assembled with the pump console 200, the start post 260 may be configured to electrically and/or mechanically engage the start button 210 of the pump console 200. In some cases, the connection/engagement between the start post 260 and the start button 210 may complete an electrical circuit or actuate a push button which allows the pump to function.

The cassette 204 may further include a lockout mechanism 264 disposed within the housing 230. Generally, the lockout mechanism 264 may be movable from a pre-use or starting position or configuration to a use position or configuration, and finally to a post-use or disabled position or configuration. Once in the disabled position, the lockout mechanism 264 mechanically prevents the cassette 204 from being fully assembled with the pump console 200 and thus prevents more than one use of the cassette 204. The lockout mechanism 264 may include a biasing mechanism 266, such as, but not limited to, a spring and a slide element 268. In some cases, the biasing mechanism 266 may be a compression spring formed from stainless steel, or another suitable material.

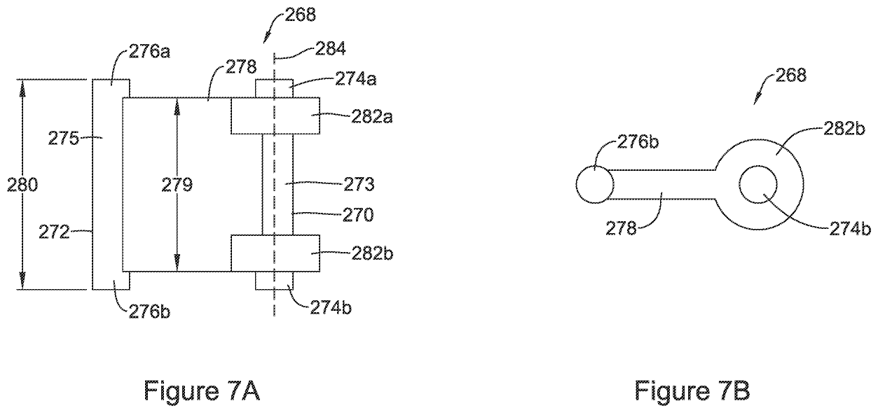

Referring additionally to FIGS. 7A and 7B which illustrate a top view and a side view, respectively, of the slide element 268; the slide element 268 may extend from a first end 270 to a second end 272. The slide element 268 may include a first rod portion 273 forming a first pair of circular protrusions 274a, 274b (collectively, 274) adjacent the first end 270 and a second rod portion 275 forming a second pair of circular protrusions 276a, 276b (collectively, 276) adjacent the second end 272 thereof. In some cases, the rod portions 273, 275 may each be formed from a cylindrical rod extending across a width of the slide element 268. As will be described in more detail herein, the protrusions 274, 276 may be configured to be disposed within and move along a slot path formed in the first and second portions 232, 234 of the housing 230. A generally planar plate 278 may extend between the first rod portion 273 and the second rod portion 275. The plate 278 may have a width 279 that is less than a width 280 of the first and second rod portions 273, 275. The slide element 268 may further include a first stabilizer 282a and a second stabilizer 282b (collectively, 282) positioned adjacent the first pair of protrusions 274. The stabilizers 282 may have a diameter that is greater than a diameter of the first pair of protrusions 274. The stabilizers 282 may be configured to allow the slide to rotate about an axis 284 of the of the first rod portion 273. Further, the stabilizers 282 may be spaced such that the outer surfaces thereof are approximately in line with the outer edge of the plate 278.

Returning to FIGS. 5 and 6, the biasing mechanism 266 may be positioned in a first channel 286. The first channel 286 may be defined by the first and second portions 232, 234 of the housing 230. The biasing mechanism 266 extends from a first end 265 to a second end 267. The first end 265 of the biasing mechanism 266 may be positioned against a wall of the housing 230 while the second end 267 of the biasing mechanism 266 may be positioned adjacent to and in contact with the first end 270 of the slide element 268. The second end 267 of the biasing mechanism 266 may be positioned between the stabilizers 282. In the pre-use configuration illustrated in FIG. 5, the slide element 268 is positioned such that slide element 268 applies a force to the biasing mechanism 266 to deform the biasing mechanism 266 from its relaxed configuration (not explicitly shown) to a compressed configuration. When the biasing force of the slide element 268 is removed the biasing mechanism 266 moves from the compressed configuration to a relaxed and expanded configuration to move the slide element 268, as will be described in more detail herein. The stabilizers 282 of the slide element 268 may have a diameter that is approximately the same as, or less than a height 285 of the channel 286. This may stabilize the slide element 268 as the slide mechanism rotates about axis 284 during insertion of the cassette 204 into a pump console 200. Further, the stabilizers 282 may facilitate loading of the biasing mechanism 266 during assembly.

The base 232 of the housing 230 includes a first slot path 287 and a second slot path 288 configured to receive a first circular protrusion 274a of the first pair of protrusions 274 and a first circular protrusion 276a of the second pair of protrusions 276. The first slot path 287 may be generally linear. The second slot path 288 may include a first or main branch portion 290 extending generally parallel to the bottom side wall 236b from a first end 289 to a second end 291. The second slot path 288 may further include a latch portion or side branch 292 extending from and connected to the main branch 290. The side branch 292 may extend at a non-orthogonal angle relative to the main branch 290. The side branch 292 may extend from a first end 293 to a connection point 294. The side branch 292 may intersect the main branch 290 at the connection point 294. The covering 234 of the housing 230 may include a mirror image first slot 281 and a mirror image second slot path 283 that is configured to receive a second circular protrusion 274b of the first pair of protrusions 274 and a second circular protrusion 276b of the second pair of protrusions 276. The second slot path 283 may include a main branch 261 and a side branch 263.

The first circular protrusion 274a of the first pair of protrusions 274 and the first circular protrusion 276a of the second pair of protrusions 276 will be used to describe the position of the slide element 268 relative to the housing 230. It should be understood the second circular protrusion 274b of the first pair of protrusions 274 and a second circular protrusion 276b of the second pair of protrusions 276 are in a similar position within the slot path 283 of the covering 234 of the housing 230. In the pre-use configuration, as shown in FIG. 5, the first protrusion 274a of the first pair of protrusions 274 is positioned at the first end 289 of the main branch 290 of the slot path 288 and the first protrusion 276a of the second pair of protrusions 276 is positioned at the first end 293 of the side branch 292 of the slot path 288. Generally, in the pre-use configuration, the slide element 268 is non-parallel to the main branch 290 of the slot path 288.

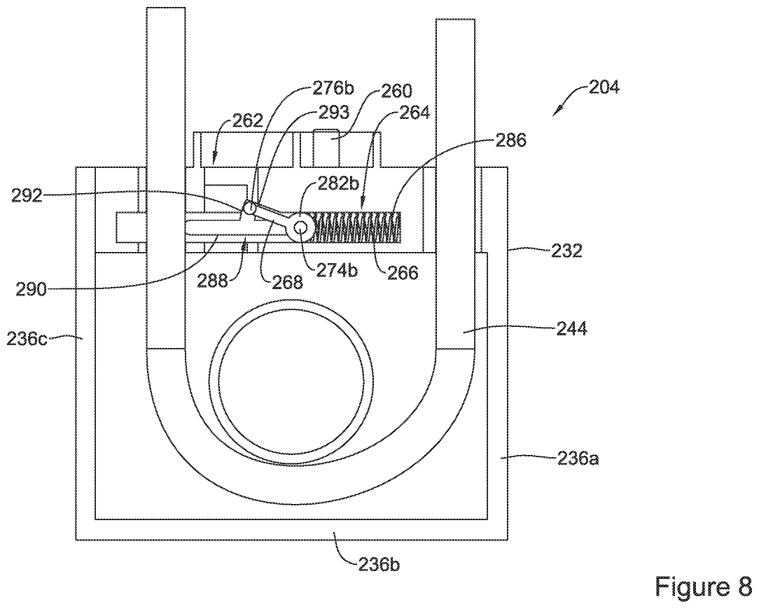

The assembly and disassembly of the cassette 204 with the pump console will be described with respect to FIGS. 8-12. In FIGS. 8-12, the covering 234 of the housing 230 of the cassette 204 is not shown to more clearly illustrate the changing configuration of the lockout mechanism 264. However, it should be understood the cassette 204 includes a cover 234. FIG. 8 illustrates a front view of the illustrative single use device or cassette 204 in a pre-use or starting configuration. As described with respect to FIG. 5, the slide element 268 is non-parallel to the main branch 290 of the slot path 288. In this configuration, the second end 272 of the slide element 268 abuts of a sidewall of the side branch 292 of the second slot path 288. The mechanical engagement of the slide element 268 with the sidewall of the second portion 292 of the second slot path 288 prevents the biasing mechanism 266 from moving the slide element 268 towards the second sidewall 236c of the covering 234 of the housing 230 and maintains the biasing mechanism 266 in the compressed pre-use configuration. When the slide element 268 is in the pre-use configuration illustrated in FIG. 8, the second end 272 of the slide element 268 extends at least partially into the insertion slot 262.

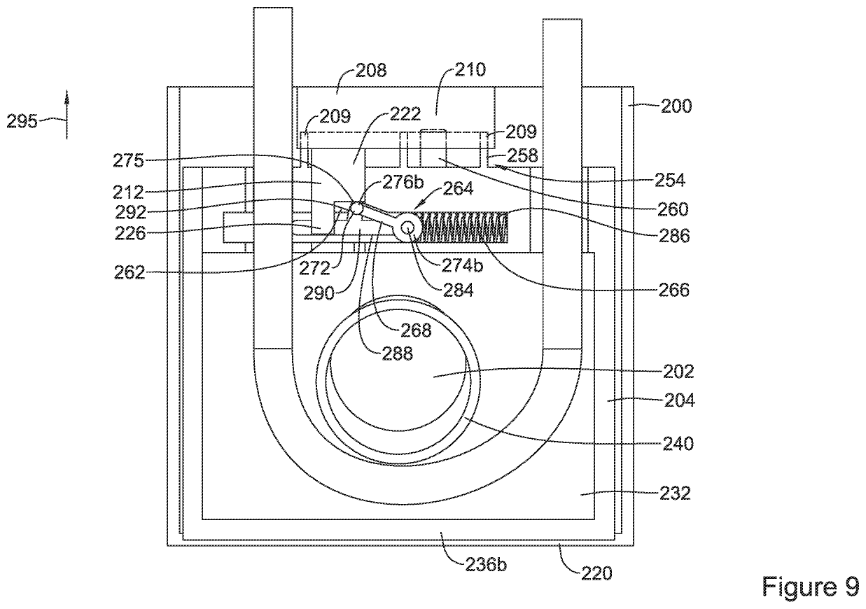

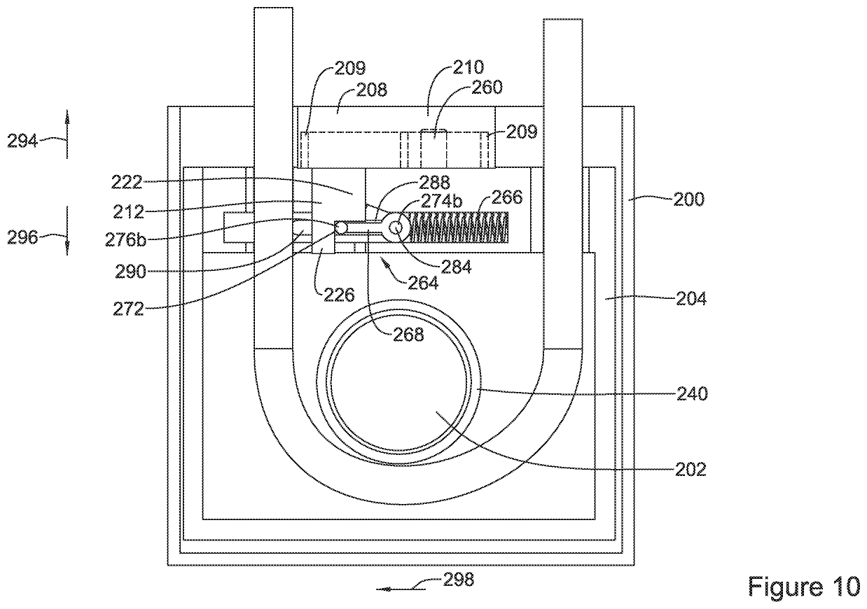

FIG. 9 illustrates a front view of the illustrative single use device or cassette 204 as it is being inserted into a pump console 200. To insert the cassette 204, the cage 254 is aligned with the mating recesses 209 of the engagement member 208 and the cassette 204 is partially inserted in the direction of arrow 295 into the pump console 200. The cassette 204 may be partially inserted into the pump console 200 at a non-parallel angle to the pump console 200. As the cassette 204 is advanced in direction 295, the insertion member 212 of the pump console 200 is received within the insertion slot 262. The second portion 226 of the insertion member 212 passes the slide element 268 without contacting the slide element 268. The first portion 222 of the insertion member 212 contacts or abuts the second rod portion 275 adjacent the second end 272 of the slide element 268. Further insertion of the cassette 204 in the direction of arrow 295 may cause the second end 272 of the slide element 268 to rotate in the direction of arrow 296 about axis 284 and into the main branch 290, as shown in FIG. 10. For example, as the second pair of protrusions 276 are disposed within the side branches 292, 263, the second end 272 of the slide element 268 may be moved along the side branches 292, 263 towards the main branch 290, 261 with continued insertion of the cassette 204 in direction 295.

The cassette 204 may be inserted until the top sidewall 236d contacts a bottom surface of the engagement member 208 of the pump console 200 to result in an engaged or in-use configuration of the cassette 204, as shown in FIG. 10. In this configuration, the cage 254 is received within the recesses 209 of the engagement member 208 such that the start post 260 of the cassette 204 electrically and/or mechanically contacts the start button 210 of the pump console 200. The connection between the start post 260 and the start button 210 may allow the pump to function. In some cases, the connection between the start button 210 and the start post 260 may activate or reset a timer. For example, a use time limit may be set at the initial mating of a new cassette 204 with the pump console 200. Connection of the start button 210 and the start post 260 may activate a timer to track usage time of the cassette 204. Once the use time limit has been reached, a new cassette 204 may be required to be inserted to reset the timer.

Further, the first portion 222 of the insertion member 212 advances the second end 272 of the slide element 268 into the main branch 290. This may remove the mechanical stop between the second end 272 of the slide element 268 and the side branch 292 allowing the biasing mechanism 266 to expand and exert a biasing force on the first end 270 of the slide element 268. The biasing force of the biasing mechanism 266 moves the slide element 268 along the main branch 290 in the direction illustrated at arrow 298. Movement of the slide element 268 is stopped by the second portion 226 of the insertion member 212 which extends through and/or past (in direction 296) the main branch 290. For example, the second end 272 of the slide element 268 rests against and is supported by the second portion 226 of the insertion member 212 creating a mechanical stop which may prevent further expansion of the biasing mechanism 266. The second end 272 of the slide element 268 may be advanced in direction 298 beyond the intersection point 294 of the main branch 290 and the side branch 292 of the slot 288. This may prevent the second end 272 of the slide element 268 from being repositioned within the side branch 292 when the cassette 204 is removed from the pump console 200.

In some cases, the housing 206 of the pump console 200 may hold the cassette 204 in place during use. In other cases, additional clamps, clips, fasteners, or locks (not explicitly shown) may be used to secure the cassette 204 within the pump console 200. As noted above, in some cases, the cassette 204 may be tilted or angled during insertion in order to mate with the console 200 (e.g., to mage with the motor shaft 202). It is contemplated the features of the pump console 200 and the cassette 204 may include sufficient tilted angle clearance.

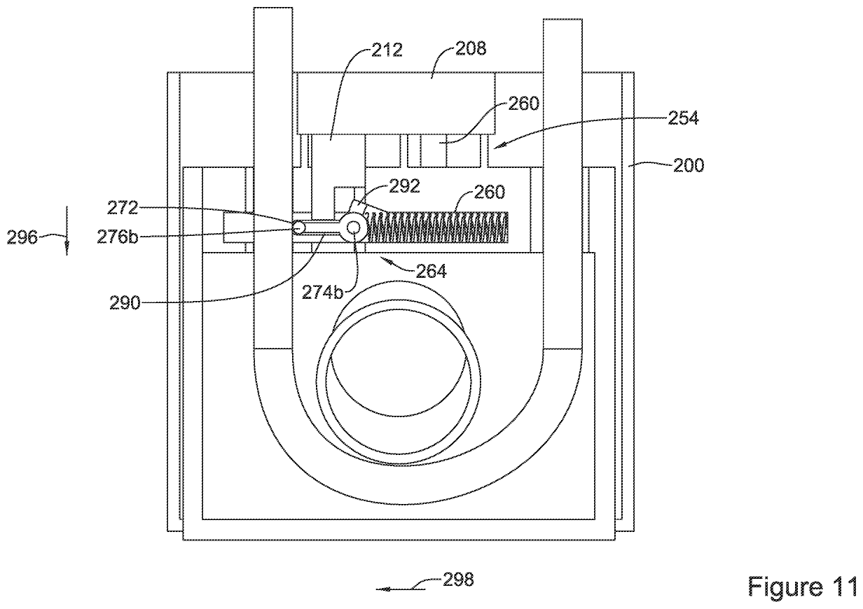

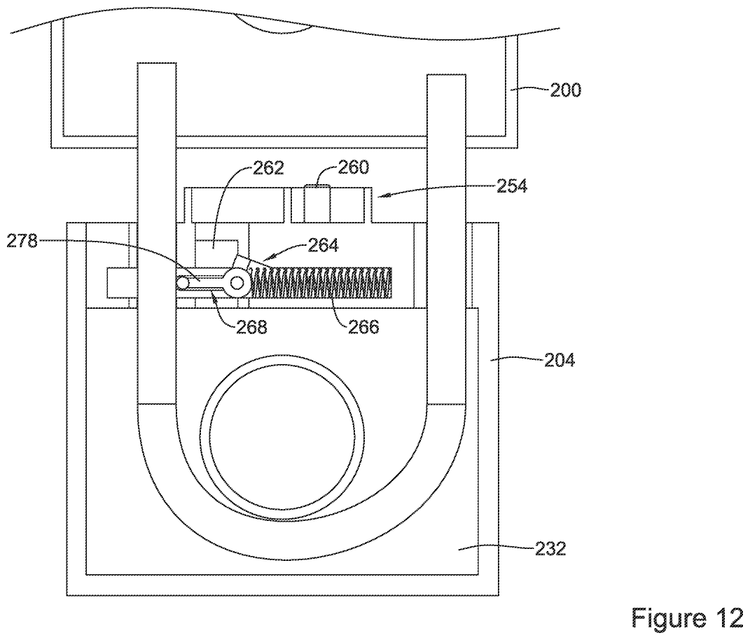

FIGS. 11 and 12 illustrate the removal of the cassette 204 from the pump console 200. In FIGS. 11 and 12, the cassette 204 is in a post-use, or disabled, configuration. To remove the cassette 204 (either at the expiration of a use time limit or completion of a procedure), the cassette 204 may be withdrawn in direction 296. As the insertion member 212 is removed from the insertion slot 262, the mechanical stop between the second end 272 of the slide element 268 and the second portion 226 of the insertion member 212 is removed allowing the biasing mechanism 266 to further expand and exert a biasing force on the first end 270 of the slide element 268. The biasing force of the biasing mechanism 266 moves the slide element 268 further along the main branch 290 in the direction illustrated at arrow 298. This may position the planar plate 278 of the slide element 268 within the insertion slot 262 which blocks or closes the insertion slot 262 such that if the previously used cassette 204 is attempted to be reinserted into a pump console 200, the plate 278 prevents the insertion member 212 from being fully inserted into the insertion slot 262. For example, as can be seen in FIG. 11, if one were to attempt to re-insert the cassette 204 into the pump console, the plate 278 blocks the insertion slot 262 such that the insertion member 212 is precluded from advancing in direction 296. This in turn may prevent the start button 210 of the pump console 200 from being engaged and/or activated and/or the motor shaft 202 and rotor (not explicitly shown) from being mated and/or the cassette 204 failing to mate with the housing 206 of the pump console 200. Said differently, the cassette 204 is prevented from properly fitting onto the pump console 200 in a functional manner after removal of the cassette 204. In some cases, the slide element 268 of the cassette 204 may be provided with a visual indicium that allows a user to quickly determine if the cassette 204 has been previously used and/or disabled. For example, the slide element 268, or portions thereof, may be colored and/or marked such that plate 278 can be viewed via the insertion slot 262 to determine if the cassette 204 has been previously used and/or disabled.

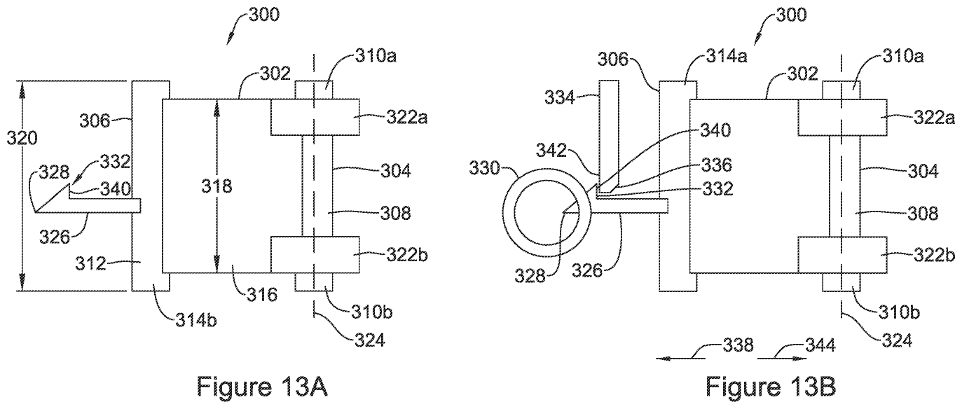

FIG. 13A illustrates a top view of another illustrative slide element 300, that may form a part of a lockout mechanism, such as, but not limited to lockout mechanism 264. FIG. 13B illustrates a top view of the illustrative slide element 300 in a post-use configuration. The slide element 300 may include a body portion 302 extending from a first end 304 to a second end 306. The body portion 302 may include a first rod portion 308 forming a first pair of circular protrusions 310a, 310b (collectively, 310) adjacent the first end 304 and a second rod portion 312 forming a second pair of circular protrusions 314a, 314b (collectively, 314) adjacent the second end 306 thereof. In some cases, the rod portions 308, 312 may each be formed from a cylindrical rod extending across a width of the body portion 302. Similar to the slide element 268 described herein, the protrusions 310, 314 may be configured to be disposed within and move along a slot path formed in the housing of a cassette. A generally planar plate 316 may extend between the first rod portion 308 and the second rod portion 312. The plate 316 may have a width 318 that is less than a width 320 of the first and second rod portions 308, 312. The slide element 300 may further include a first stabilizer 322a and a second stabilizer 322b (collectively, 322) positioned adjacent the first pair of protrusions 306. The stabilizers 322 may have a diameter that is greater than a diameter of the first pair of protrusions 306. The stabilizers 322 may be sized and shaped to allow the slide to rotate about an axis 324 of the of the first rod portion 308. Further, the stabilizers 322 may be spaced such that the outer surfaces thereof are approximately in line with the outer edge of the plate 316.

The slide element 300 may further include a tip 326 extending from the second end 306 of the body portion 302. The tip 326 may have a distal end 328 that is sharp, blunt, a hook, etc. For example, the distal end 328 may be configured to disable the flexible tube 330 of a cassette. In some cases, a distal end 328 may be configured to disable components of the cassette in addition to, or in place of the flexible tube 330. In one example, a sharp distal end 328 may be configured to damage and/or pierce the flexible tube 330. In another example, a blunt distal end 328 may be configured to crimp, kink, or otherwise damage or break the flexible tube 330. Damaging the flexible tube 330, or another component of the cassette, may render the cassette non-functional. It is contemplated that this may provide an additional mechanism for preventing reuse of a cassette. For example, if the slide element 300 was somehow to be repositioned to the pre-use configuration, the damaged components of the cassette may prevent the cassette from functioning properly.

In some examples, the distal end 328 may include a hook or latch 332. The hook 332 may be configured to engage a rib 334 or other protruding feature of the cassette to lock the slide element 300 in the post-use configuration. For example, the rib 334 may include an angled surface 336 which allows the hook 332 to slide past the rib 334 in a first direction 338. However, a ledge 340 of the hook 332 may engage a surface 342 of the rib 334 to create a mechanical stop which prevents the slide element 300 from moving in a second direction 344 opposite the first direction 338.

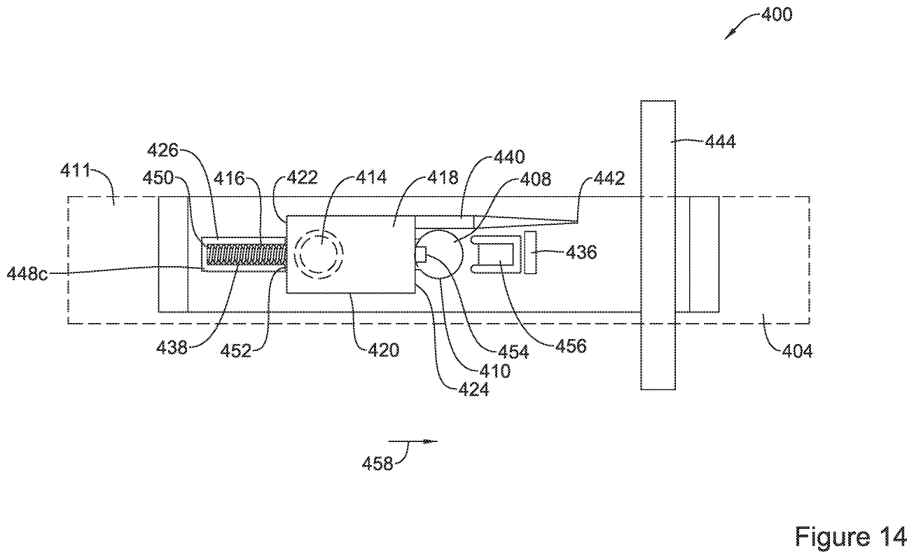

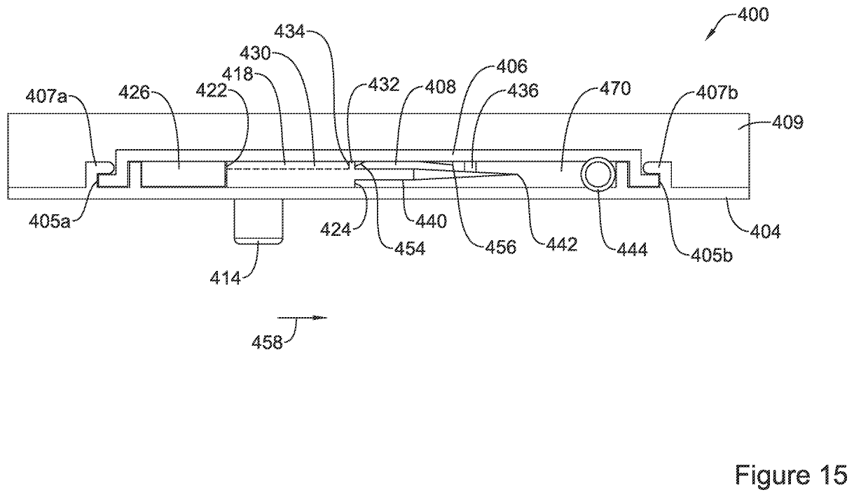

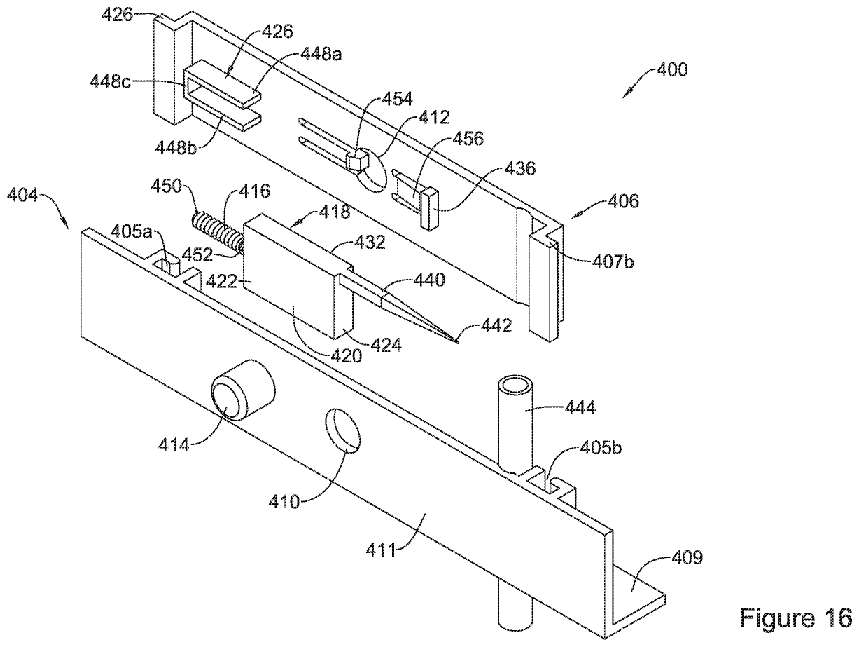

FIG. 14 is a schematic side view of another illustrative lockout mechanism 400. FIG. 15 is a schematic top view of the illustrative lockout mechanism 400 of FIG. 14. FIG. 16 is an exploded perspective view of the illustrative lockout mechanism 400 of FIG. 14. While not explicitly shown, the lockout mechanism 400 may be positioned within or form a part of a single use device, such as, but not limited to a cassette, similar in form and function to the cassette 204 described herein. The lockout mechanism 400 may include a front housing 404 and a back plate 406. The front housing 404 may include a first groove 405a and a second groove 405b (collectively, 405). The back plate 406 may include a first tongue 407a and a second tongue 407b (collectively, 407). The tongues 407 may be sized and shaped to mate with the grooves 405 to form an interlocking connection. The front housing 404 may further include a lower plate 409 extending generally perpendicular to a front plate 411. When assembled, a bottom edge of the back plate 406 may rest on the lower plate 409 of the front housing 404. In some cases, the front housing 404 and/or the back plate 406 may form a part of a housing of a cassette, although this is not required. In some cases, the front housing 404 and/or the back plate 406 may be positioned within a housing of the cassette. When a cassette including the lockout mechanism 400 is assembled with a pump console, the front housing 404 may be positioned towards the housing of the pump console.

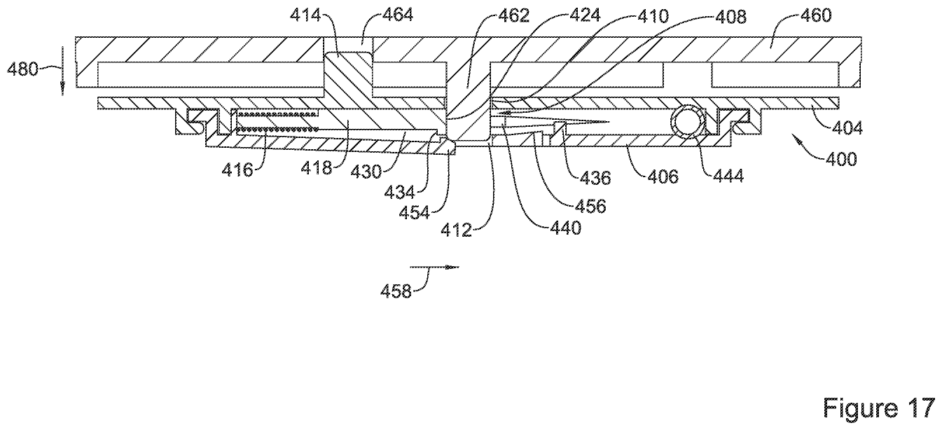

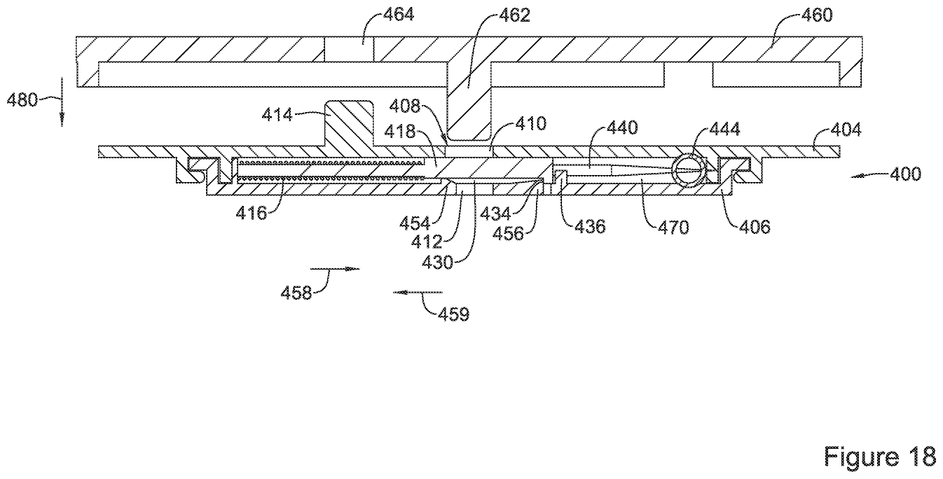

The lockout mechanism 400 may include an insertion slot 408 configured to receive a mating insertion member 462 (see, for example, FIGS. 17 and 18) on the pump console 460. The insertion slot 408 may include a first aperture 410 formed in the front plate 411 of the front housing 404 and a second aperture 412 formed in the back plate 406. The first and second apertures 410, 412 may be generally aligned to allow the insertion member 462 of the pump console 460 to pass through both the first and second apertures 410, 412. It is contemplated that the insertion slot 408 may be positioned such that assembly of the cassette with the pump console 460 is limited to a predetermined orientation.

The lockout mechanism 400 may further include a start post 414 extending from front housing 404 in a direction away from the back plate 406. When the cassette is fully assembled with the pump console 460, the start post 414 may be configured to electrically and/or mechanically engage the start button 464 of the pump console 460 in a manner similar to that described above. In some cases, the connection between the start post 414 and the start button 464 may complete an electrical circuit or actuate a push button which allows the pump to function. Further, actuation of the start button 464 may activate a timer, as described above.

Generally, the lockout mechanism 400 may be movable from a pre-use or starting position or configuration to a use position or configuration, and finally to a post-use or disabled position or configuration. Once in the disabled position, the lockout mechanism 400 mechanically prevents the cassette from being fully assembled with a pump console and thus prevents more than one use of the cassette in which the lockout mechanism 400 is contained. The lockout mechanism 400 may include a biasing mechanism 416, such as, but not limited to, a spring, and a slide element 418. In some cases, the biasing mechanism 416 may be a compression spring formed from stainless steel, or another suitable material.

The slide element 418 may include a body portion 420 extending from a first end 422 to a second end 424. The body portion 420 may have a generally rectangular cross-section having a size and shape configured to be positioned between the front housing 404 and the back plate 406. In some cases, the body portion 420 may include a recess 430 formed in a lateral side 432 thereof. The recess 430 may create a shelf or ledge 434 which is configured to engage a mating stop 436 on the back plate 406 when the lockout mechanism 400 is in the post-use or disabled configuration.

In some cases, the slide element 418 may include an elongate shaft 438 extending from the first end thereof. In some cases, the elongate shaft 438 may be received within a lumen of the biasing mechanism 416. However, in some embodiments, the elongate shaft 438 may be omitted. The slide element 418 may further include a tip 440 extending from the second end 424 of the body portion 420. The tip 440 may have a distal end 442 that is sharp, blunt, a hook, etc. For example, the distal end 442 may be configured to disable the flexible tube 444 of a cassette. In some cases, a distal end 442 may be configured to disable components of the cassette in addition to, or in place of the flexible tube 444. In one example, a sharp distal end 442 may be configured to damage and/or pierce the flexible tube 444. In another example, a blunt distal end 442 may be configured to crimp, kink, or otherwise damage or break the flexible tube 444. Damaging the flexible tube 444, or another component of the cassette, may render the cassette non-functional. It is contemplated that this may provide an additional mechanism for preventing reuse of a cassette. For example, if the slide element 418 was somehow to be repositioned to the pre-use configuration, the damaged components of the cassette may prevent the cassette from functioning properly. While not explicitly shown, in some examples, the distal end 442 may include a hook or latch configured to engage a rib or other protruding feature of the cassette or lock out mechanism 400 to lock the slide element 418 in the post-use configuration. It is further contemplated that in some examples, the tip 440 may be omitted.

The biasing mechanism 416 may be positioned in a biasing mechanism or spring housing 426. The biasing mechanism housing 426 may be defined by the front housing 404 and the back plate 406. In some cases, the back plate 406 may include one or walls 448a, 448b, 448c (collectively, 448) extending from a surface thereof that together with the front housing 404 form the biasing mechanism housing 426. The biasing mechanism 416 extends from a first end 450 to a second end 452. The first end 450 of the biasing mechanism 416 may be positioned against a wall 448c of the back plate 406 while the second end 452 of the biasing mechanism 416 may be positioned adjacent to and in contact with the first end 422 of the slide element 418. In the pre-use configuration illustrated in FIGS. 14 and 15, the slide element 418 is positioned such that the slide element 418 applies a force to the biasing mechanism 416 to deform the biasing mechanism 416 from its relaxed configuration (not explicitly shown) to a compressed configuration. For example, the second end 424 of the body portion 420 may engage a latch 454 formed in the back plate 406 adjacent to and extending into the aperture 412 which prevents movement of the slide element 418 in direction 458. When the biasing force of the slide element 418 is removed, the biasing mechanism 416 moves from the compressed configuration to a relaxed and expanded configuration to move the lockout mechanism 400, as will be described in more detail herein. For example, the latch 454 may be deflectable such that when the insertion member 462 of the pump console 460 is inserted into the insertion slot 408, the latch 454 is deflected or moved out of the path of the slide element 418.

The back plate 406 may further include a cantilever ramp 456 which may lock or maintain the lockout mechanism 400 in the disabled configuration after use of the cassette. The cantilever ramp 456 may extend into a channel 470 formed by the front housing 404 and the back plate 406. The protrusion or stop 436 may also extend from the back plate 406 and into the channel 470 of the lockout mechanism 400. The stop 436 may be configured to limit movement of the slide element 418 in direction 458 when the latch 454 is released.

The assembly and disassembly of the cassette and lockout mechanism 400 with a pump console 460 will be described with respect to FIGS. 17-18. In FIGS. 17-18, the details of the cassette and portions of the pump console are not shown to more clearly illustrate the changing configuration of the lockout mechanism 400. FIG. 17 illustrates a partial cross-sectional view of the lockout mechanism 400 assembled with a pump console 460 with the lockout mechanism in a use configuration.

The cassette may be inserted into the pump console 460 such that the insertion slot 408 aligns with the insertion member 462 of the pump console 460. In the illustrated embodiment, the insertion slot 408 has a generally circular cross-section and the insertion member 462 is sized and shaped to fit therethrough. However, it is contemplated that the insertion slot 408 may take any cross-sectional shape desired and the insertion member 462 shaped to mate therewith. The insertion member 462 is advanced through the first aperture 410 in the front housing 404 and at least partially through the second aperture 412 in the back plate 406. It is contemplated that the length of the insertion member 462 may be long enough to deflect the latch 454 which extends partially into the second aperture 412. The insertion member 462 may move the latch 454 away from the back plate 406 in direction 480 and out of the path of the second end 424 of the slide element 418. Upon removal of the mechanical stop between the latch 454 and the ledge 434 of the slide element 418, the biasing mechanism 416 may expand and move the slide element 418 along the channel 470 in direction 458. The slide element 418 may move under the force of the biasing mechanism 416 until the second end 424 of the slide element 418 contacts the insertion member 462. For example, the second end 424 of the slide element 418 rests against and is supported by the insertion member 462 creating a mechanical stop which may prevent further expansion of the biasing mechanism 416. When so provided, the tip 440 of the slide element 418 may be positioned above or below the insertion slot 408 (see, for example, FIG. 14) such that the tip 440 does not interfere with the insertion member 462 or so that the insertion member 462 does not prevent the tip 440 from advancing.

In the in-use configuration shown in FIG. 17, the start post 414 of the lockout mechanism 400 electrically and/or mechanically contacts the start button 464 of the pump console 460. The connection between the start post 414 and the start button 464 may allow the pump to function. In some cases, activation of the start button 464 may initiate a timer, as described above. Further, in the in-use configuration, the second end 424 of the body portion 420 of the slide element 418 may be advanced in direction 458 beyond the latch 454. This may prevent ledge 434 and/or the second end 424 of the body portion 420 of the slide element 418 from being re-engaged with the latch 454 when the cassette is removed from the pump console 460.

FIG. 18 is a cross-sectional view of the illustrative lockout mechanism 400 of FIG. 14 in a post-use configuration with a pump console 460. To remove the cassette, the cassette may be withdrawn in direction 480. As the insertion member 462 is removed from the insertion slot 408, the mechanical stop between the second end 424 of the slide element 418 and the insertion member 462 is removed allowing the biasing mechanism 416 to further expand and exert a biasing force on the first end 422 of the slide element 418. The biasing force of the biasing mechanism 416 moves the slide element 418 further along the channel 470 in the direction illustrated at arrow 458. The second end 424 of the slide element 418 may engage the protrusion 436 to prevent further movement in direction 459. Further, the ledge 434 may engage the cantilever ramp to prevent the slide element 418 from moving backward in direction 459.

In the post-use configuration, the body portion 420 of the slide element 418 may be positioned within the insertion slot 408. This may block or close the insertion slot 408 such that if the previously used cassette is attempted to be reinserted into a pump console 460, the body portion 420 prevents the insertion member 462 from being fully inserted into the insertion slot 408. This in turn may prevent the start button 464 of the pump console 460 from being engaged and/or activated and/or the motor shaft and rotor (not explicitly shown) from being mated and/or the cassette failing to mate with the housing of the pump console 460. Said differently, the cassette is prevented from properly fitting onto or within the pump console 460 in a functional manner after removal of the cassette. In some cases, the slide element 418 of the lockout mechanism 400 may be provided with a visual indicium that allows a user to quickly determine if the cassette has been previously used and/or disabled. For example, the slide element 418, or portions thereof, may be colored and/or marked such that body portion 420 can be viewed via the insertion slot 408 to determine if the cassette has been previously used and/or disabled.

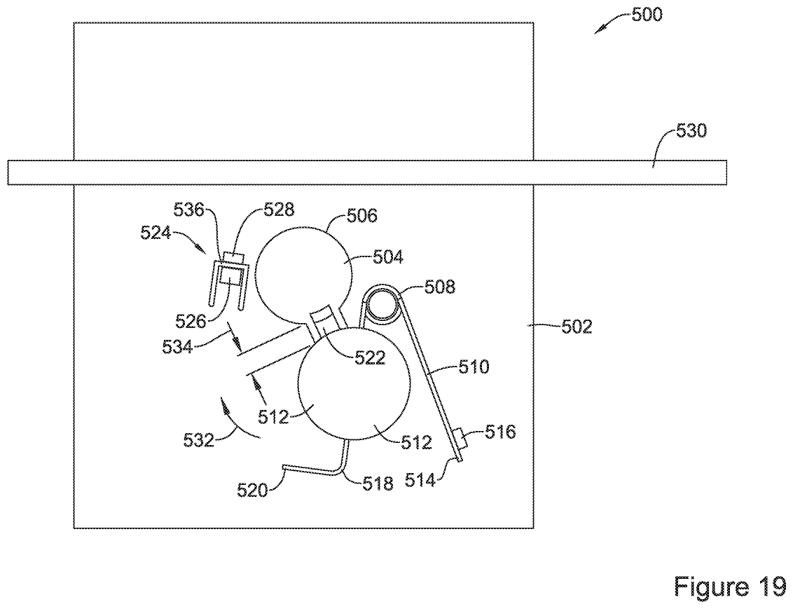

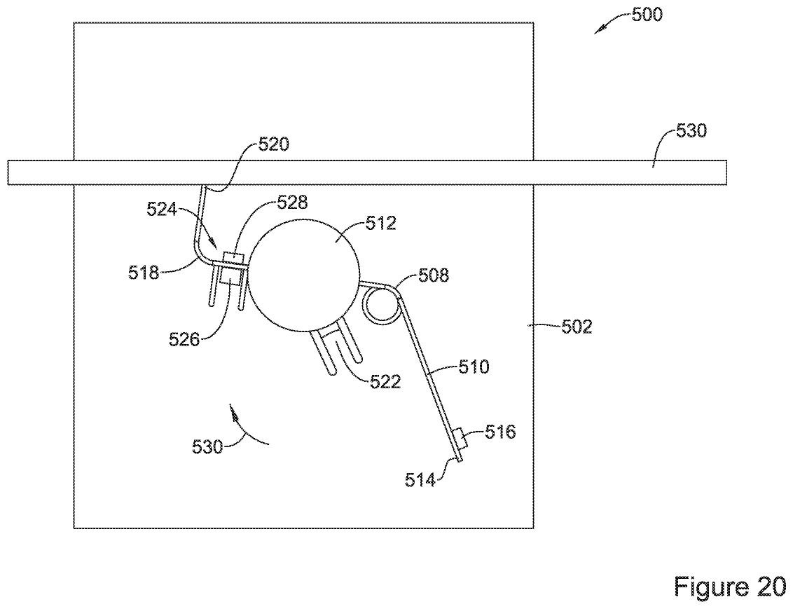

FIG. 19 is a schematic side view of another illustrative lockout mechanism 500 in a pre-use configuration. FIG. 20 is a schematic side view of the illustrative lockout mechanism 500 of FIG. 19 in a post-use or disabled configuration. While not explicitly shown, the lockout mechanism 500 may be positioned within or form a part of a single use device, such as, but not limited to a cassette, similar in form and function to cassette 204 described herein. The lockout mechanism 500 may include a front housing (not explicitly shown) and a back housing 502. In some cases, the front housing and/or the back housing 502 may form a part of a housing of a cassette, although this is not required. In some cases, the front housing and/or the back housing 502 may be positioned within a housing of the cassette. When a cassette including the lockout mechanism 500 is assembled with a pump console, the front housing may be positioned towards the housing of the pump console.

The lockout mechanism 500 may include an insertion slot 504 configured to receive a mating insertion member (not explicitly shown) on the pump console. The insertion slot 504 may include an aperture 506 formed in the back housing 502. In some cases, an aperture may be formed in the front housing and aligned with the aperture 506 in the back housing 502. It is contemplated that the insertion slot 504 may be positioned such that assembly of the cassette with the pump console is limited to a predetermined orientation.

While not explicitly shown, the lockout mechanism 500 may further include a start post. When the cassette is fully assembled with the pump console, the start post may be configured to electrically and/or mechanically engage a start button of the pump console in a manner similar to that described above. In some cases, the connection between the start post and the start button may complete an electrical circuit or actuate a push button which allows the pump to function. Further, actuation of the start button may activate a timer, as described above.

Generally, the lockout mechanism 500 may be movable from a pre-use or starting position or configuration (FIG. 19) to a use position or configuration (not explicitly shown), and finally to a post-use or disabled position or configuration (FIG. 20). Once in the disabled position, the lockout mechanism 500 mechanically prevents the cassette from being fully assembled with a pump console and thus prevents more than one use of the cassette in which the lockout mechanism 500 is contained. The lockout mechanism 500 may include a biasing mechanism 510, such as, but not limited to, a spring, and a slide element 512. In some cases, the biasing mechanism 510 may be a torsion spring formed from stainless steel, or another suitable material. The biasing mechanism 510 may be configured to rotate the slide element 512 about an axle 508 to move the slide element 512 from a pre-use configuration (FIG. 19), to an in-use configuration (not explicitly shown), and finally a post use configuration (FIG. 20). The biasing mechanism 510 may include a first leg 514 which may be secured to the back housing 502 (and/or the front housing) via an anchor block 516. The anchor block 516 may prevent the first leg 514 of the biasing mechanism 510 from moving. It is further contemplated that the axle 508 may be secured to the back housing 502 (and/or the front housing) in a manner that allows a second leg 518 to rotate while the axle 508 remains in a similar position.

The slide element 512 may include a generally planar disk sized and shaped to at least partially block the insertion slot 504. In some cases, a cross-sectional dimension of the slide element 512 may be about the same, smaller than, or greater than a cross-sectional dimension of the insertion slot 504. The slide element 512 may be maintained in the pre-use configuration via an articulatable latch 522. For example, the latch 522 may mechanically engage an edge of the slide element 512. In the pre-use configuration illustrated in FIG. 19, the slide element 512 is positioned such that the slide element 512 applies a force to the biasing mechanism 510 to deform the biasing mechanism 510 from its relaxed configuration (FIG. 20) to a compressed configuration (FIG. 19). For example, the slide element 512 may engage a latch 522 formed in the back housing 502 adjacent to and extending into the insertion slot 504 which prevents movement of the slide element 512. When the biasing force of the slide element 512 is removed, the biasing mechanism 510 moves from the compressed configuration to a relaxed and expanded configuration to move the lockout mechanism 500, as will be described in more detail herein. For example, the latch 522 may be deflectable such that when the insertion member of the pump console is inserted into the insertion slot 504, the latch 522 is deflected or moved out of the path of the slide element 512. The back housing 502 may further include a cantilever ramp 524 which may lock or maintain the lockout mechanism 500 in the disabled configuration after use of the cassette. The cantilever ramp 524 may include a ramp portion 526 and a ledge 528.

To assemble a cassette including lockout mechanism 500, the cassette may be positioned into the pump console such that the back housing 502 of the lockout mechanism 500 faces away from the pump console and the front housing is towards the pump console. The cassette may be inserted into the pump console such that the insertion slot 504 aligns with an insertion member of the pump console. In the illustrated embodiment, the insertion slot 504 has a generally circular cross-section and the insertion member may be sized and shaped to fit therethrough. However, it is contemplated that the insertion slot 504 may take any cross-sectional shape desired and the insertion member shaped to mate therewith.

The insertion member may be advanced through an aperture in the front housing and at least partially through the aperture 506 in the back housing 502. It is contemplated that the length of the insertion member may be long enough to deflect the latch 522 which extends partially into the aperture 506. The insertion member may move the latch 522 away from the back housing 502 in direction opposite from the front housing and out of the path of the slide element 512. Upon removal of the mechanical stop between the latch 522 and the slide element 512, the biasing mechanism 510 may expand and rotate the slide element 512 as shown at arrow 532. The slide element 512 may rotate under the force of the biasing mechanism 510 an edge of the slide element 512 contacts the insertion member. For example, the slide element 512 may move a distance 534 until an edge of the slide element 512 contacts the insertion member.

FIG. 20 is a schematic side view of the illustrative lockout mechanism 500 of FIG. 19 in a post-use or disabled configuration. To remove the cassette, the cassette may be withdrawn in the opposite direction of insertion. As the insertion member is removed from the insertion slot 504, the mechanical stop between the slide element 512 and the insertion member is removed allowing the biasing mechanism 510 to further expand and rotate the slide element 512, as shown at arrow 532. This may position the slide element 512 within the insertion slot 504. This may block or close the insertion slot 504 such that if the previously used cassette is attempted to be reinserted into a pump console, the slide element 512 prevents the insertion member from being fully inserted into the insertion slot 504. This in turn may prevent the start button of the pump console from being engaged and/or activated and/or the motor shaft and rotor (not explicitly shown) from being mated and/or the cassette failing to mate with the housing of the pump console. Said differently, the cassette is prevented from properly fitting onto or within the pump console in a functional manner after removal of the cassette. In some cases, the slide element 512 of the lockout mechanism 500 may be provided with a visual indicium that allows a user to quickly determine if the cassette has been previously used and/or disabled. For example, the slide element 512, or portions thereof, may be colored and/or marked such that slide element can be viewed via the insertion slot 504 to determine if the cassette has been previously used and/or disabled.

As the slide element 512 is rotated towards the disabled configuration, the second leg 518 of the biasing mechanism 510 may be guided up and over the ramp portion 526 of the lock ramp 524. The second leg 518 may then lower to rest in a groove 536 between a lock stop 528 and against the back side of the ramp portion 526. This mechanical interlock may prevent the biasing mechanism 510 from unintentional repositioning into the pre-use configuration or from further rotation of the slide mechanism in direction 532.

In some cases, the second leg 518 of the biasing mechanism 510 may further include a tip portion 520. In some cases, the tip portion 520 may be an extension of the biasing mechanism 510. The tip portion 520 may be angled or otherwise positioned to come into contact with a flexible tube 530 of the cassette when the lockout mechanism 500 is in the disabled configuration. For example, the tip portion 520 may be configured to puncture or disable the flexible tube 530 of a cassette. In some cases, the tip portion 520 may be configured to disable components of the cassette in addition to, or in place of the flexible tube 530. In one example, a sharp tip portion 520 may be configured to damage and/or pierce the flexible tube 530. In another example, a blunt tip portion 520 may be configured to crimp, kink, or otherwise damage or break the flexible tube 530. Damaging the flexible tube 530, or another component of the cassette, may render the cassette non-functional. It is contemplated that this may provide an additional mechanism for preventing reuse of a cassette. For example, if the slide element 512 was somehow to be repositioned to the pre-use configuration, the damaged components of the cassette may prevent the cassette from functioning properly. While not explicitly shown, in some examples, the tip portion 520 may include a hook or latch configured to engage a rib or other protruding feature of the cassette or lockout mechanism 500 to lock the slide element 512 in the post-use configuration. It is further contemplated that in some examples, the tip portion 520 may be omitted.

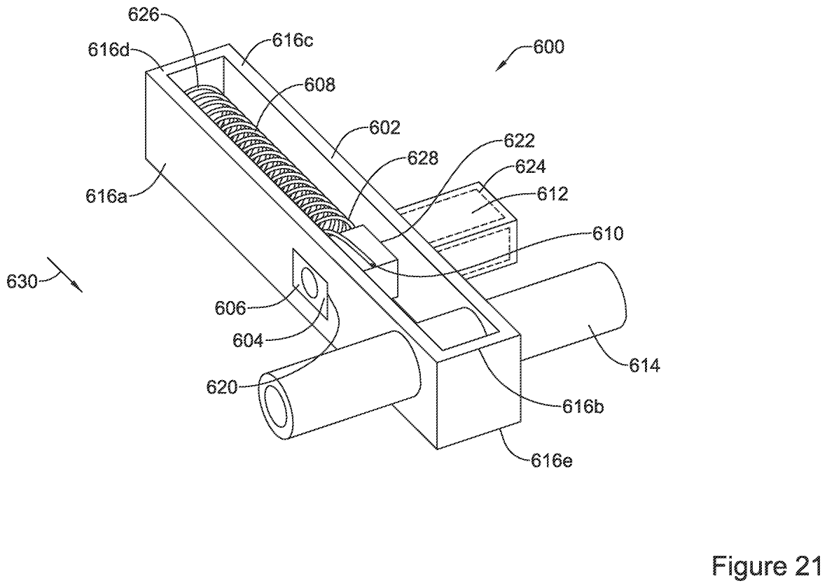

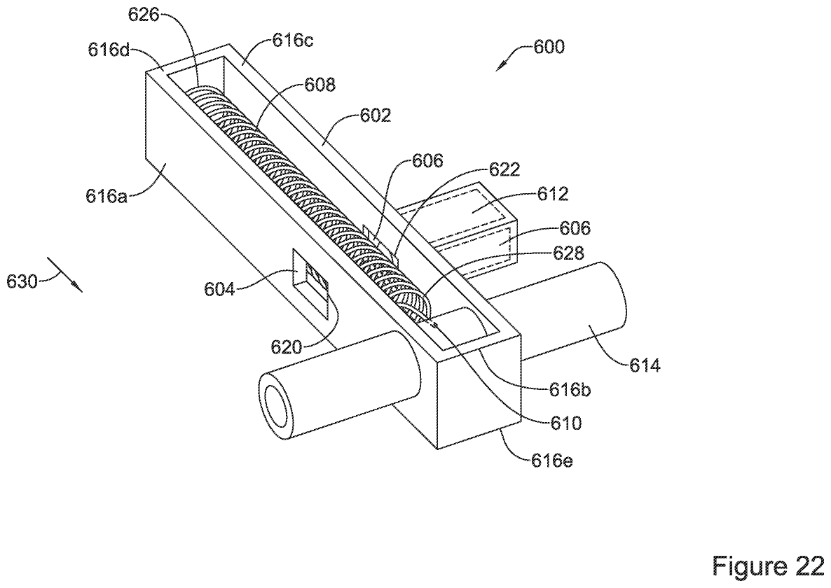

FIG. 21 is a perspective side view of another illustrative lockout mechanism 600 in a pre-use configuration. FIG. 22 is a perspective view of the illustrative lockout mechanism 600 of FIG. 21 in a post-use or disabled configuration. While not explicitly shown, the lockout mechanism 600 may be positioned within or form a part of a single use device, such as, but not limited to a cassette, similar in form and function to cassette 204 described herein. The lockout mechanism 600 may include housing 602. The housing 602 may generally take the form of a rectangular prism including a plurality of side walls 616a, 616b, 616c, 616d, 616e (collectively, 616) defining a cavity 618. While not explicitly shown, the housing 602 may additionally include a top wall to fully enclose the cavity 618. In some cases, the housing 602 may form a part of a housing of a cassette, although this is not required. In some cases, the housing 602 may be positioned within a housing of the cassette. When a cassette including the lockout mechanism 600 is assembled with a pump console, the back sidewall 616e may be positioned towards the housing of the pump console, although this is not required.

The lockout mechanism 600 may include an insertion slot 604 configured to receive a mating insertion member (not explicitly shown) on the pump console. The insertion slot 604 may extend from a first opening 620 in a first lateral sidewall 616a to a second opening 622 in a second lateral sidewall 616c. The insertion slot 604 may include a slide element or latch 606 disposed therethrough. In some cases, the latch 606 may at least partially extend into the openings 620, 622 such that the latch 606 rests on an edge of the openings 620, 622 and is suspended between the sidewalls 616a, 616c. The latch 606 may be a molded block or an extruded tube (square, round, oval profile or the like) that has a slight interface fit with the insertion slot 604 at opening 620 and a taper or a rounded edge at the end closest to the second opening 622.

It is contemplated that the insertion slot 604 may be positioned such that assembly of the cassette with the pump console is limited to a predetermined orientation. The lockout mechanism 600 may further include a displacement box 612 positioned adjacent to and in communication with the second opening 622. The displacement box 612 may define a cavity 624 that is sized and shaped to receive the latch 606.

While not explicitly shown, the lockout mechanism 600 may further include a start post. When the cassette is fully assembled with the pump console, the start post may be configured to electrically and/or mechanically engage a start button of the pump console in a manner similar to that described above. In some cases, the connection between the start post and the start button may complete an electrical circuit or actuate a push button which allows the pump to function. Further, actuation of the start button may activate a timer, as described above.

Generally, the lockout mechanism 600 may be movable from a pre-use or starting position or configuration (FIG. 21) to a use position or configuration (not explicitly shown), and finally to a post-use or disabled position or configuration (FIG. 22). Once in the disabled position, the lockout mechanism 600 mechanically prevents the cassette from being fully assembled with a pump console and thus prevents more than one use of the cassette in which the lockout mechanism 600 is contained. The lockout mechanism 600 may include a biasing mechanism 608, such as, but not limited to, a spring. In some cases, the biasing mechanism 608 may be a compression spring formed from stainless steel, or another suitable material.

The biasing mechanism 608 may be positioned in the cavity 618 of the housing 602 such at that a first end 626 of the biasing mechanism 608 is positioned against a lateral sidewall 616d of the housing 602. In the pre-use configuration illustrated in FIG. 21, the biasing mechanism 608 is maintained in a compressed configuration due to a mechanical engagement of a second end 628 of the biasing mechanism 608 with the latch 606. When the biasing force of the latch 606 is removed the biasing mechanism 608 moves from the compressed configuration to a relaxed and expanded configuration (FIG. 22). For example, the biasing mechanism 608 may be configured to elongate from a compressed pre-use configuration (FIG. 21), to an intermediate in-use configuration (not explicitly shown), and finally an elongated post-use configuration (FIG. 22). The biasing mechanism 608 may further include a tip 610 extending from the second end 628 thereof. As will be described in more detail herein, the tip 510 may be configured to disable a flexible tube 614 or other component of the cassette.

To assemble a cassette including lockout mechanism 600, the cassette may be positioned into the pump console such that the insertion slot 604 is aligned with an insertion member of the pump console. In the illustrated embodiment, the insertion slot 604 has a generally rectangular cross-section and the insertion member may be sized and shaped to fit therethrough. However, it is contemplated that the insertion slot 604 may take any cross-sectional shape desired and the insertion member shaped to mate therewith.

The insertion member may be advanced through the first opening 620 and at least partially through the second opening 622. It is contemplated that the length of the insertion member may be long enough to displace the latch 606. For example, as the insertion member is advanced into the insertion slot 604, the latch 606 is pushed into the cavity 624 of the displacement box 612. Upon removal of the mechanical stop between the latch 606 and the biasing mechanism 608, the biasing mechanism 608 may come into contact with the insertion member to form an in-use configuration. In some cases, the biasing mechanism 608 may at least partially elongate to contact the insertion member, although this is not required.

FIG. 22 is a perspective view of the illustrative lockout mechanism 600 of FIG. 21 in a post-use or disabled configuration. To remove the cassette, the cassette may be withdrawn in the opposite direction of insertion. As the insertion member is removed from the insertion slot 604, the mechanical stop between the biasing mechanism 608 and the insertion member is removed allowing the biasing mechanism 608 to further expand and elongate, as shown at arrow 630. This may position the biasing mechanism 608 within the insertion slot 604. This may block or close the insertion slot 604 such that if the previously used cassette is attempted to be reinserted into a pump console, the biasing mechanism 608 prevents the insertion member from being fully inserted into the insertion slot 604. This in turn may prevent the start button of the pump console from being engaged and/or activated and/or the motor shaft and rotor (not explicitly shown) from being mated and/or the cassette failing to mate with the housing of the pump console. Said differently, the cassette is prevented from properly fitting onto or within the pump console in a functional manner after removal of the cassette. In some cases, the biasing mechanism 608 of the locking mechanism 600 may be provided with a visual indicium that allows a user to quickly determine if the cassette has been previously used and/or disabled. For example, biasing mechanism 608, or portions thereof, may be colored and/or marked such that slide element can be viewed via the insertion slot 604 to determine if the cassette has been previously used and/or disabled.

In some cases, the biasing mechanism 608 may further include a tip 610. In some cases, the tip 610 may be an extension of the biasing mechanism 608. The tip 610 may be sized and shaped to come into contact with a flexible tube 614 of the cassette when the lockout mechanism 600 is in the disabled configuration. For example, the tip 610 may be configured to puncture or disable the flexible tube 614 of a cassette. In some cases, the tip 610 may be configured to disable components of the cassette in addition to, or in place of the flexible tube 614. In one example, a sharp tip 610 may be configured to damage and/or pierce the flexible tube 614. In another example, a blunt tip 610 may be configured to crimp, kink, or otherwise damage or break the flexible tube 614. Damaging the flexible tube 614, or another component of the cassette, may render the cassette non-functional. It is contemplated that this may provide an additional mechanism for preventing reuse of a cassette. For example, if the biasing mechanism 608 was somehow to be repositioned to the pre-use configuration, the damaged components of the cassette may prevent the cassette from functioning properly. While not explicitly shown, in some examples, the tip 610 may include a hook or latch configured to engage a rib or other protruding feature of the cassette or lock out mechanism 600 to lock the biasing mechanism 608 in the post-use configuration. It is further contemplated that in some examples, the tip 610 may be omitted.

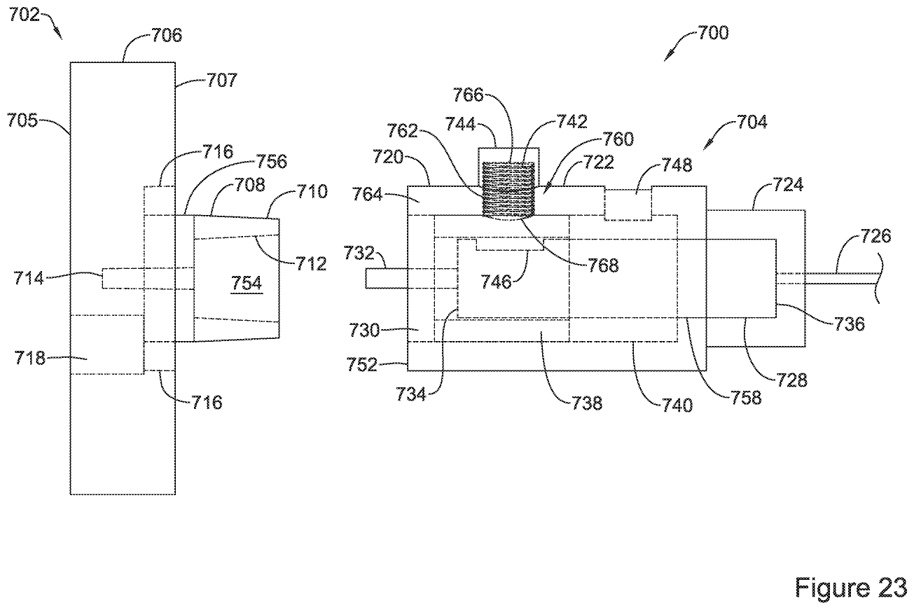

As described above, the lockout mechanisms described herein are not limited to cassettes and pump consoles. The lockout mechanisms may be used in other single use devices as well. FIG. 23 is a side view of an illustrative single use laser fiber assembly 700 in a pre-use configuration. While the assembly 700 is described with respect to a single use laser fiber, other single use devices, such as, but not limited to, electrocautery plugs, electric plugs, optical plugs, fluid plugs, single use scope shafts, etc. may be provided in place of the laser fiber. The assembly may include a console 702 and a laser fiber device 704.

The console 702 may include a housing 706 and an insertion member 708. In some cases, the housing 706 may have a generally cylindrical shape, although this is not required. The insertion member 708 may be a generally tubular structure extending distally from a distal side 707 of the housing 706. The insertion member 708 may have a proximal portion 756 having a generally uniform outer diameter and a distal portion 710 having an outer diameter that tapers or reduces in the distal direction. The distal portion 710 of the insertion member 708 may including an inner surface 712 defining a cavity 754 therein. A diameter of the inner surface 712 may increase in the distal direction. A female connector slot 714 may extend proximally from the cavity 754, through the proximal portion 756 and into the housing 706.

An annular recess 716 generally surrounding the insertion member 708 may be formed in the distal side 707 of the housing 706. Said differently, the insertion member 708 may protrude from a central region of the annular recess 716. The annular recess 716 may be configured to receive a portion of the laser fiber device 704, as will be described in more detail herein. The housing 706 may further include a micro switch or starter switch 718 positioned within the housing 706. The micro switch 718 may extend distally from a proximal end 705 towards the annular recess 716. A portion distal end of the micro switch 718 may extend into the annular recess 716 or form a part of a proximal wall of the recess 716.

The laser fiber device 704 may include a laser fiber housing 728 extending from a proximal end 734 to a distal end 736. A laser fiber 726 may extend distally from the distal end 736 of the laser fiber housing 728. The laser fiber housing 728 may have a generally circular outer diameter. In some cases, the laser fiber housing 728 may include a generally flat recess 746 formed in an outer surface thereof. The recess 746 may be configured to receive a component of a lockout mechanism, as will be described herein. Further, a male connector 732 may extend proximally from the proximal end 734 of the laser fiber housing 728.

A lockout mechanism housing 720 may be disposed over the laser fiber housing 728. Said differently, the laser fiber housing 728 may disposed within a cavity 730 of the lockout mechanism housing 720. The lockout mechanism housing 720 may include a proximal portion 722 having a first outer diameter and a distal portion 724 having a second outer diameter less than the first outer diameter. However, this is not required. In some cases, the first and second outer diameters may be similar or the same or the second outer diameter may be greater than the first outer diameter. The distal end 736 of the laser fiber housing 728 may be disposed within the distal portion 724 of the lockout mechanism housing 720 while the proximal end 734 of the laser fiber housing 728 may be disposed within the proximal portion 722 of the lockout mechanism housing 720.

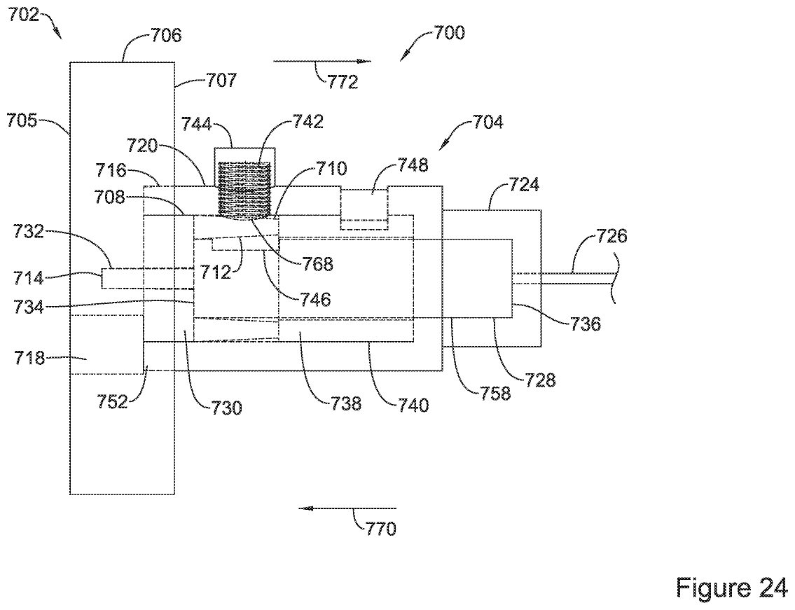

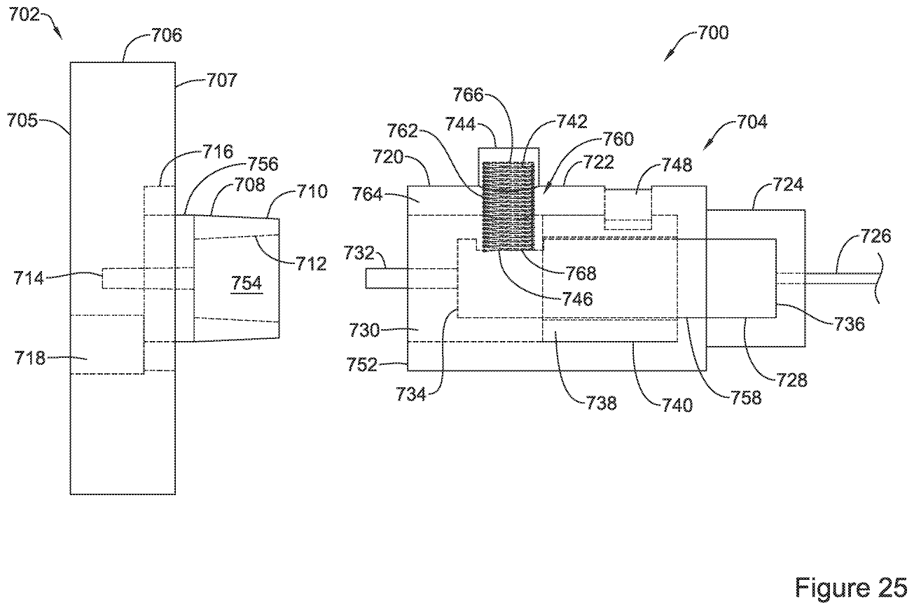

The lockout mechanism housing 720 may further include a lockout mechanism 760. Generally, the lockout mechanism 760 may be movable from a pre-use or starting position or configuration (FIG. 23) to a use position or configuration (FIG. 24), and finally to a post-use or disabled position or configuration (FIG. 25). Once in the disabled position, the lockout mechanism 760 mechanically prevents the laser fiber device 704 from being fully assembled with the console 702 and thus prevents more than one use of the laser fiber device 704 in which the lockout mechanism 760 is contained. The lockout mechanism 760 may include a slide element or latch 738 and a biasing mechanism or spring element 742. The slide element 738 may have a generally tubular shape that is disposed between an inner wall 740 of the proximal portion 722 of the lockout mechanism housing 720 and an outer surface 758 of the laser fiber housing 728. An inner diameter of the slide element 738 may be approximately the same as or greater than the outer diameter of the laser fiber housing 728. This may allow the slide element 738 to move longitudinally relative to the laser fiber housing 728. The slide element 738 may have an outer diameter that is approximately the same as or less than the inner diameter of the proximal portion 722 of the lockout mechanism housing 720 such that the slide element 738 can move longitudinally relative to the lockout mechanism housing 720. In some cases, the outer diameter and/or the inner diameter of the slide element 738 may form a slight interference fit with the lockout mechanism housing 720 or the laser fiber housing 728, respectively, to prevent unintentional movement of the slide element 738. While not explicitly shown, in some cases, a distal end region of the slide element 738 may be tapered to facilitate longitudinal movement of the slide element 738 in the distal direction.

The lockout mechanism housing 720 may further include a radially extending spring element housing 744 which is generally aligned with the recess 746 of the laser fiber housing 728. The spring element housing 744 may define a cavity therein for receiving the spring element 742. The spring element 742 may be a compression spring, a disk spring, a wave spring, elastic member, or the like. The spring element 742 may extend from the cavity of the spring element housing 744, through an aperture 762 in a sidewall 764 of the proximal portion 722 of the lockout mechanism housing 720 and into the cavity 730 of the lockout mechanism housing 720. The spring element 742 may extend from a first end 766 to a second end 768. The spring element 742 may be positioned in the cavity of the spring element housing 744 such at that a first end 766 of the spring element 742 is positioned against a lateral sidewall of the housing 744. In the pre-use configuration illustrated in FIG. 23, the spring element 742 is maintained in a compressed configuration due to a mechanical engagement of a second end 768 of the spring element 742 with the outer surface of the slide element 738. In some cases, the force of the spring element 742 may help prevent unintentional movement of the slide element 738. When the biasing force of the slide element 738 is removed the spring element 742 moves from the compressed configuration to a more elongate configuration. For example, the spring element 742 may be configured to elongate from a compressed pre-use configuration (FIG. 23) to an intermediate in-use configuration (FIG. 24), and finally an elongated post-use configuration (FIG. 25).

In some cases, the lockout mechanism housing 720 may include an opening or window 748 extending through the sidewall 764 of the proximal portion 722. The window 748 may extend through a thickness of the sidewall 764 such that a user can see into the cavity 730. As will be described in more detail herein, the cavity 730 may allow a user to determine if the slide element 738 has been actuated.

While not explicitly shown, the laser fiber housing 728 may include a flat slot or slot that extends about a perimeter thereof. The slot may facilitate coupling of the laser fiber housing 728 to the lockout mechanism housing 720. In some cases, the laser fiber housing 728 may be coupled to the lockout mechanism housing 720 adjacent to a distal end 736 of the laser fiber housing 728, although this is not required. It is contemplated that the laser fiber housing 728 may be fixedly coupled to the lockout mechanism housing 720 through the use of glues or adhesives or mechanism means, such as, but not limited to, crimping, mechanism fasteners, etc.

FIG. 24 is a side view of the illustrative single use laser fiber of FIG. 23 in a use configuration. To assemble the laser fiber device 704 with the console 702, the lockout mechanism housing 720 is aligned with the insertion member 708 and inserted onto the insertion member 708 in direction 770. The proximal end 752 of the lockout mechanism housing 720 is disposed with the annular recess 716. While the lockout mechanism housing 720 and the recess 716 are generally described as circular or annular, other shapes may be used as desired. Circular shapes may allow for rotation of the lockout mechanism housing 720 relative to the insertion member 708 when mater. Other shapes, such as rectangular, oval, hexagon, polygonal, etc. may be used to preclude rotation.

As the insertion member 708 is advanced into the cavity 730 of the lockout mechanism housing 720, the distal portion 710 of the insertion member 708 contacts the slide element 738 and advances the slide element 738 in the distal direction, as shown at arrow 772. The distal portion 710 may be sized and shaped such that the proximal end 734 of the laser fiber housing 728 is disposed within the cavity 754 of the insertion member 708. Movement of the slide element 738 allows the second end 768 of the spring element 742 to extend onto the outer surface of the distal portion 710 of the insertion member 708. In the fully seated configuration, the distal end of the insertion member 708 extends distally beyond the spring element 742. When the laser fiber device 704 is fully seated in the console 702, shown in FIG. 24, the slide element 738 is longitudinally displaced to a distal end region of the proximal portion 722 of the lockout mechanism housing 720. The slide element 738 may be provided with a visual indicium that allows a user to quickly determine if the laser fiber device 704 has been previously used and/or disabled. For example, slide element 738, or portions thereof, may be colored and/or marked such that slide element 738 can be viewed via the window748 to determine if the cassette has been previously used and/or disabled.

Further, when the laser fiber device 704 is fully seated with the console 702, the proximal end 752 of the lockout mechanism housing 720 may depress or otherwise activate the micro switch 718. This may allow the procedure to begin. In some cases, actuation of the micro switch 718 may also activate and/or reset a timer. The timer may include a predetermined use time limit for the single use device. Once the predetermined use time has elapsed the console 702 may no longer allow the laser fiber device 704 to function. Thus, a new laser fiber device 704 may be required to reset the timer and continue the procedure. In the fully seated configuration, the male connector 732 is received within the female connector slot 714. This may electrically and/or mechanically couple the laser fiber 726 to the console 702.

To uncouple the laser fiber device 704 from the console 702, the laser fiber device 704 is advanced in the distal direction 772 relative to the console 702. FIG. 25 is a side view of the illustrative single use laser fiber of FIG. 23 in a post-use configuration. As the laser fiber device 704 is distally advanced the insertion member 708 of the console 702 is proximally retracted from the cavity 730 of the lockout mechanism housing 720. Removal of the insertion member 708 may allow the spring element 742 to expand. In the expanded configuration, the second end of the spring element 742 is disposed within the recess 746 of the laser fiber housing 728. In this configuration, the spring element 742 blocks the insertion slot or path of the insertion member 708 and prevents the full seating of the laser fiber device 704 with the console 702. For example, when the insertion member 708 cannot fully advance within the cavity 730, the male connector 732 of the laser fiber 726 cannot be fully received within the female connector slot 714 preventing function of the laser fiber 726. Further, when the insertion member 708 cannot fully advance within the cavity 730 the proximal end 752 of the lockout mechanism housing 720 fails to activate the micro switch 718.

The lockout mechanisms 264, 300, 400, 500, 600, 760, the components thereof, and/or the insertion members 212, 462, 708 configured to activate the lockout mechanisms 264, 300, 400, 500, 600, 760 may be formed in a number of different ways, including but not limited to, additive manufacturing, stamping, machining, molding, injection molding, etc.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A single use medical device, comprising:
a housing defining an insertion slot; and
a lockout mechanism comprising:
a biasing mechanism; and
a movable slide element positioned adjacent to the insertion slot and the biasing mechanism, the slide element movable between a pre-use configuration, an in-use configuration, and a post-use configuration;
wherein the slide element is actuated via an insertion member removably inserted into the insertion slot.

2. The single use medical device of claim 1, wherein when the slide element is in the post-use configuration, the slide element is positioned at least partially within the insertion slot.

3. The single use medical device of claim 1, wherein when the slide element is in the pre-use configuration, the slide element mechanically engages a portion of the housing.

4. The single use medical device of claim 1, wherein when the slide element is in the in-use configuration, the slide element mechanically engages the insertion member

5. The single use medical device of claim 1, wherein the biasing mechanism is configured to exert a force on the slide element.

6. The single use medical device of claim 5, wherein in the absence of a stop mechanism, the biasing mechanism is configured to bias the slide element to the post-use configuration.

7. The single use medical device of claim 1, wherein when the slide element is in the post-use configuration, the slide element is visible through the insertion slot.

8. The single use medical device of claim 1, further comprising a start post, the start post configured to engage a start button on a multi-use device.

9. The single use medical device of claim 1, further comprising an elongated tip extending from an end of the slide element.

10. The single use medical device of claim 9, wherein the elongated tip is configured to disable a component of the single use device when the slide element is in the post-use configuration.

11. The single use medical device of claim 1, wherein insertion of the insertion member is configured to move the slide element from the pre-use configuration to the in-use configuration.

12. The single use medical device of claim 1, wherein the biasing mechanism comprises a compression spring.

13. The single use medical device of claim 1, wherein the biasing mechanism comprises a torsion spring.

14. The single use medical device of claim 1, wherein the single use device comprises a cassette for use in a fluid management system.

15. The single use medical device of claim 1, wherein the single use device comprises a laser fiber, an electrocautery plug, an electric plug, an optical plug, a fluid plug, or a single use scope shaft.

16. A medical device system, comprising:
a multi-use device, comprising:
a housing defining a cavity; and
an insertion member; and
a single use device configured to be removably coupled with the multi-use device, the single use device, comprising:
a housing defining an insertion slot; and
a lockout mechanism comprising:
a biasing mechanism; and
a movable slide element positioned adjacent to the insertion slot and the biasing mechanism, the slide element movable between a pre-use configuration, an in-use configuration, and a post-use configuration;
wherein when the slide element is in the post-use configuration, the single use device is prevented from being functionally assembled with the multi-use device.

17. The medical device system of claim 16, wherein insertion of the insertion member into the insertion slot is configured to move the slide element from the pre-use configuration to the in-use configuration and subsequent removal of the insertion member from the insertion slot is configured to move the slide element form the in-use configuration to the post-use configuration.

18. A medical device system, comprising:
a multi-use device, comprising:
a housing defining a cavity;
an insertion member; and
a start button; and
a single use device configured to be removably coupled with the multi-use device, the single use device, comprising:
a housing defining an insertion slot;
a start post; and
a lockout mechanism comprising:
a biasing mechanism; and
a movable slide element positioned adjacent to the insertion slot and the biasing mechanism, the slide element movable between a pre-use configuration, an in-use configuration, and a post-use configuration;
wherein the slide element is moved from the pre-use configuration to the in-use configuration upon insertion of the insertion member into the insertion slot; and
wherein when the slide element is in the post-use configuration, the single use device is prevented from being functionally assembled with the multi-use device.

19. The medical device system of claim 18, wherein when the slide element is in the in-use configuration, the start post of the single use device engages the start button of the multi-use device.

20. The medical device system of claim 19, wherein when the start post engages the start button, a timer is activated.
